(19) United States
(12) Patent Application Publication
(10) Pub. No.: US 2018/0098728 A1
(43) Pub. Date: Apr. 12, 2018

(54) NON-INVASIVE METHOD FOR ASSESSING THE PRESENCE OR SEVERITY OF LIVER FIBROSIS BASED ON A NEW DETAILED CLASSIFICATION

(71) Applicants: CENTRE HOSPITALIER UNIVERSITAIRE D'ANGERS, Angers (FR); UNIVERSITE D'ANGERS, Angers (FR)

(72) Inventors: Paul CALES, Avrille (FR); Jerome BOURSIER, Angers (FR)

(21) Appl. No.: 15/787,962

(22) Filed: Oct. 19, 2017

Related U.S. Application Data
(63) Continuation-in-part of application No. 14/004,608, filed on Sep. 11, 2013, now abandoned.

(30) Foreign Application Priority Data
Mar. 11, 2011 (EP) 11157978.5

(51) Int. Cl. A61B 5/00

(52) U.S. Cl. CPC A61B 5/4842; A61B 5/4244

(57) ABSTRACT

The present invention relates to for treating for an individual identified as suffering from a liver lesion, preferably liver fibrosis or cirrhosis. The present invention thus relates to a method invention for implementing an adapted patient care for an individual suffering from a liver lesion, preferably liver fibrosis or cirrhosis, said method including the steps of (i) determining in the individual the presence and severity of a liver lesion, preferably liver fibrosis or cirrhosis, by carrying out at least one non-invasive test resulting in a value and positioning the at least one value in a class of a detailed classification; and (ii) implementing an adapted patient care for the individual depending on the severity of the liver lesion, preferably liver fibrosis or cirrhosis, as determined by the class of the detailed classification wherein the at least one value was positioned.

### FIELD OF INVENTION

The present invention relates to a non-invasive method for assessing the presence and/or the severity of liver fibrosis or cirrhosis. More specifically, the present invention relates to a non-invasive method implementing a new detailed classification of liver fibrosis stages, leading to an improved diagnostic accuracy and precision. The present invention further relates to a method for implementing an adapted patient care for an individual suffering from a liver lesion, preferably liver fibrosis or cirrhosis, depending on the severity of said liver lesion, preferably liver fibrosis or cirrhosis.

### BACKGROUND OF INVENTION

Liver fibrosis refers to the accumulation in the liver of fibrous scar tissue in response to injury of the hepatocytes due to various etiologies, such as for examples infection with a virus (such as hepatitis viruses HCV and HBV), heavy alcohol consumption, toxins or trauma. The evolution of the fibrosis phenomena may lead to cirrhosis, a condition in which the ability of the liver to function is impaired. Treatments of liver fibrosis exist, which can slow or halt fibrosis progression, and even reverse existing liver damages. On the contrary, cirrhosis is usually non-reversible. Therefore, the earlier the diagnostic of a fibrosis is, the more elevated the chances of reversion are.

Liver biopsy is the historical means in order to diagnose liver diseases in patients. Various systems, based on liver biopsies, are used to grade fibrosis and cirrhosis, such as, for example, Metavir, NASH-CRN and Ishak (where cirrhosis is graded). Using Metavir scoring system for fibrosis, five classes (named Metavir F stages) are distinguished: F0 (no fibrosis, no scarring), F1 (portal fibrosis, minimal scarring), F2 (few septa, scarring has occurred and extents outside the areas in the liver that contains blood vessels), F3 (many septa, bridging fibrosis is spreading and connecting to other areas that contain fibrosis) and finally F4 (cirrhosis or advanced scarring of the liver). Using NASH-CRN scoring system, in particular for patients suffering from NAFLD (non-alcoholic fatty liver disease), five classes are distinguished: F0 (no fibrosis), F1 (perisinusoidal or portal/periportal fibrosis), F2 (perisinusoidal and portal/periportal fibrosis), F3 (bridging fibrosis) and finally F4 (cirrhosis). Fibrosis of stages Metavir F3 or F4 is considered as “severe fibrosis”. For patients with “clinically significant fibrosis” (i.e., with Metavir score ≥F2), a treatment is usually recommended, whereas patients with no or mild fibrosis (F0 or F1 Metavir score) do not usually receive any treatment, but are monitored for fibrosis progression. Fibrosis of stage F≥3 according to the NASH-CRN scoring system is considered “advanced fibrosis”, especially in patients with NAFLD. In patients with NASH (non-alcoholic steatohepatitis), preferably with a NAFLD Activity Score (NAS)≥4, fibrosis of stage F≥2 according to the NASH-CRN scoring system characterizes the NASH as a fibrotic NASH. Ranging a patient according to the Metavir classification or the NASH-CRN scoring system helps for determining the adapted treatment for said patient. In this patent application, any citation of F0, F1, F2, F3 and F4 is made with reference either to the Metavir stages or to the NASH-CRN scoring system.

When using Metavir scoring system for assessing necrotico-inflammatory activity, four grades (named Metavir A grades) are distinguished: A0 (absence of necrotico-inflammatory activity), A1 (low necrotico-inflammatory activity), A2 (moderate necrotico-inflammatory activity), and A3 (high necrotico-inflammatory activity). In this patent application, any citation of A0, A1, A2, A3 is made with reference to the Metavir grades.

However, since liver biopsy is invasive and expensive, non-invasive diagnosis of liver fibrosis has gained considerable attention over the last 10 years as an alternative to liver biopsy. The first generation of simple blood fibrosis tests combined common indirect blood markers into a simple ratio, like APRI (Wai et al., Hepatology 2003) or FIB-4 (Sterling et al., Hepatology 2006). The second generation of calculated tests combined indirect and/or direct fibrosis markers by logistic regression, leading to a score, like Fibrotest (Imbert-Bismut et al., Lancet 2001), ELF score (Rosenberg et al., Gastroenterology 2004), FibroMeter™ (Cales et al., Hepatology 2005), Fibrospect™ (Patel et al., J Hepatology 2004), and Hepascore (Adams et al., Clin Chem 2005). For example, WO2005/116901 describes a non-invasive method for assessing the presence of a liver disease and its severity, by measuring levels of specific variables, including biological variables and clinical variables, and combining said variables into mathematical functions, generally binary mathematical function to provide a score result, often called “score of fibrosis”.

Also, in the prior art the sequential algorithm for fibrosis evaluation (SAFE) and the Bordeaux algorithm (BA), which cross-check FibroTest with the aspartate aminotransferase-to-platelet ratio index (APRI) or FibroScan (also known as Vibration Controlled Transient Elastography or VCTE), are very accurate but provide only a binary diagnosis of significant fibrosis (SAFE or BA for Metavir F≥2) or cirrhosis (SAFE or BA for F4). Therefore, in clinical practice, physicians have to apply the algorithm for Metavir F≥2, and then, when needed, the algorithm for Metavir F4 (“successive algorithms”).

For statistical reasons, these tests were constructed as a result of a mathematical function and included two classes of fibrosis stages. For example, they allow the diagnostic of the presence of significant fibrosis, with the classes Metavir F0/1 (absence of significant fibrosis) and Metavir F2/3/4 (presence of significant fibrosis). Another example is the diagnostic of cirrhosis, with the classes Metavir F0/1/2/3 (absence of cirrhosis) and Metavir F4 (presence of cirrhosis). The currently most accurate tests present an accuracy of about 75% of correctly classified patients regarding significant fibrosis. However, due to the existing 25% of misclassified patients, a biopsy remains regularly prescribed for patients suspected with severe fibrosis in order to confirm the diagnostic, especially in the indeterminate zone.

There is thus a need for an improved non-invasive method leading to a higher diagnostic accuracy and also, very important precision (increasing the number of fibrosis classes, with comparison to the mathematical binary function, above two), in order to lower or discard the need of liver biopsy. Consequently, there is a need for a method where the precision/accuracy ratio is satisfactory, i.e., leads to a low need or to no need of biopsy. Also, there is a need for a method with a low discrepancy degree.

In order to improve the possibility of distinguishing several fibrosis stages and/or necrotico-inflammatory activity grades, better than with a single mathematical function, a statistical analysis using discriminant analysis and/or polynomial logistic regression was proposed. This lead to a classification with 5 classes or more, but the classification accuracy was insufficient or even low at about 50% compared to about 75% for a binary diagnosis.

The Inventors described a non-invasive method (hereinafter referred to “2008 RDI Method”), adapted from FibroMeter™ analysis and involving the measure of RDI (Reliable Diagnosis Interval, Cales et al., Liver International, 2008). This method usually combines the diagnostic cut-off of a binary diagnosis with the thresholds of 90 to 95% predictive values of a test, resulting in a classification with four classes, namely F0/1, F1/2, F1/2/3, F2/3/4, and presenting a high accuracy (89.5% of well classified patients). However, these RDIs lead to broad classes, where it is unclear in what extent the patient has to be treated, and the need of biopsy may remain.

Now, the Inventors propose a new invention overcoming the drawbacks of the prior art, which is an improved non-invasive method for assessing the presence and/or the severity of lesions, such as, for example, liver fibrosis, based on a new detailed classification and resulting in the ability of ranging or sorting the patient more precisely according to the fibrosis stage, with reference either to the Metavir system or to the NASH-CRN scoring system, and as or more accurately than with the 2008 RDI Method.

### SUMMARY

The present invention thus relates to a non-invasive method for assessing the presence and/or severity of a lesion in an organ of an animal (including a human), said method comprising carrying out at least one non-invasive test resulting in a value, and positioning the at least one value in a class, preferably a diagnostic class, of a detailed classification based on population percentiles, or in a reliable diagnostic interval (RDI), to be crossed with another RDI for a final positioning of both RDIs in a class, preferably in a diagnostic class.

Another object of the invention is a non-invasive method for assessing the presence and/or severity of a lesion in an organ of an animal, including a human, said method comprising the steps of:
(a) carrying out at least one non-invasive test resulting in a value, preferably said value is a score result,
(b) positioning the at least one value in a class of a detailed classification, and
(c) assessing the presence and/or the severity of a lesion in an organ based on the class wherein said score result has been positioned in step (b).

According to an embodiment, the animal is a mammal, such as, for example, a rat or a pet, such as, for example, a cat or a dog. According to a preferred embodiment, the animal is a human.

According to an embodiment, the organ is the liver and the detailed classification is a detailed fibrosis classification and/or a detailed necrotico-inflammatory activity classification. In one embodiment, the detailed classification is a detailed fibrosis classification, wherein each class corresponds to less than or equal to 3 pathological fibrosis stages, such as, for example, Metavir F stages. In one embodiment, the detailed classification is a detailed fibrosis classification wherein each class corresponds to less than or equal to 3 pathological activity grades, such as, for example, Metavir A grades.

According to an embodiment, the animal, including a human, is at risk of suffering or is suffering from a condition selected from the group comprising a liver impairment, a chronic liver disease, a hepatitis viral infection especially an infection caused by hepatitis B, C or D virus, a hepatotoxicity, a liver cancer, a steatosis, a non-alcoholic fatty liver disease (NAFLD), a non-alcoholic steatohepatitis (NASH), an autoimmune disease, a metabolic liver disease and a disease with secondary involvement of the liver.

According to an embodiment, hepatotoxicity is alcohol induced hepatotoxicity and/or drug-induced hepatotoxicity (i.e., any xenobiotic compound like alcohol or drug).

According to an embodiment, autoimmune disease is selected from the group consisting of autoimmune hepatitis 5 (AIH), primary biliary cirrhosis (PBC) and primary sclerosing cholangitis (PSC).

According to an embodiment, metabolic liver disease is selected from the group consisting of hemochromatosis, Wilson's disease and alpha 1 anti-trypsin deficiency.

According to an embodiment, said disease with a secondary involvement of the liver is celiac disease or amyloidosis.

In one embodiment, the detailed classification is based on population (generally patient population) percentiles. In another embodiment, the detailed classification is based on the combination of at least two reliable diagnostic intervals (RDIs).

In one embodiment, a liver biopsy is needed after carrying out the non-invasive method in less than 30% of the classified patients.

In one embodiment, the detailed classification of the invention presents:

1. a discrepancy score lower than or equal to 0.4; and/or
2. a proportion of significant discrepancies lower than or equal to 20; and/or
3. a precision/accuracy ratio ranging from 1 to less than 5; and/or
4. a precision/accuracy/liver biopsy ratio lower than or equal to 7.

According to an embodiment, the non-invasive test comprises at least one combination score result, obtained by a mathematical combination, preferably by logistic regression or by synchronous binary combination, of at least one biomarker, at least one clinical marker, at least one data resulting from a physical method and/or at least one score result.

Advantageously, said combination score is a test selected from the group comprising ELF, FIBROSpect™, APRI, FIB-4, Hepascore, Fibrotest™, FibroMeter™ and CirrhoMeter™, preferably said non-invasive score is a FibroMeter^{3G}. In one embodiment, ELF is a blood test based on hyaluronic acid, P3P, TIMP-1 and age; FIBROSpect™ is a blood test based on hyaluronic acid, TIMP-1 and A2M; APRI is a blood test based on platelet and AST; FIB-4 is a blood test based on platelet, ASAT, ALT and age; Hepascore is a blood test based on hyaluronic acid, bilirubin, alpha2-macroglobulin, GGT, age and sex Fibrotest™ is a blood test based on alpha2-macroglobulin, haptoglobin, apolipoprotein A1, total bilirubin, GGT, age and sex; FibroMeter™ and CirrhoMeter™ are a blood test based on alpha2-macroglobulin, hyaluronic acid, prothrombin index, platelets, ASAT, ALAT, Urea, GGT, bilirubin, ferritin, glucose, age and/or sex.

In an embodiment of the invention, said physical method is selected from the group comprising medical imaging data, preferably is selected from the group comprising ultrasonography, especially Doppler-ultrasonography, elastometry ultrasonography and velocimetry ultrasonography, such as, for example, Fibroscan™ also known as Vibration Controlled Transient Elastography (VCTE), Acoustic Radiation Force Impulse (ARFI), VTE; IRM; and MNR, especially MNR elastometry or velocimetry, more preferably the physical method is Fibroscan™.

According to an embodiment of the invention, the method of the invention comprises carrying out at least one non-invasive test resulting in a value, which may be a score result or an imaging data, and positioning the at least one value in a class of a detailed fibrosis and/or activity classification based on percentiles, wherein said classification is based on the discretization of the score results of a reference population into at least 10 percentiles of 10% of the population, preferably into at least 20 percentiles of 5% of the population, more preferably into 40 percentiles of 2.5% of the population (or more, 50 percentiles of 2% of the population, 100 percentiles of 1% of the population . . . ), followed by determination of thresholds, and formation of blocks.

In an embodiment, the method of the invention comprises the steps of performing at least two non-invasive tests resulting in at least two values, which may be at least two score results, or at least one score result and at least one imaging data, or at least two imaging data.

Advantageously, said at least two non-invasive tests are FibroMeter™ and Fibroscan™ also known as Vibration Controlled Transient Elastography.

According to an embodiment, the non-invasive method of the invention comprises the steps of:
combining the values obtained from two non-invasive tests in at least two binary logistic regressions to obtain at least two indexes (value from 0 to 1),
positioning each index on a RDI, determined from a reference population according to the RDI2008 method,
combining both RDIs according to a double entry table of RDIs showing combined classes. An example of such a double entry table is given below:

| | | RDI of first Index | | | |
|---|---|---|---|---|---|
| | | Class W | Class X | Class Y | Class Z |
| RDI of second | Class A | Class AW | Class AX | Class AY | Class AZ |
| Index | Class B | Class BW | Class BX | Class BY | Class BZ |
| | Class C | Class CW | Class CX | Class CY | Class CZ |

positioning the RDI classes in combined classes (such as, in the above table, classes AW to CZ).

The present invention also refers to a device carrying out the non-invasive method of the invention.

According to an embodiment, the device is a meter reflecting the detailed classification, such as, for example, the new detailed fibrosis stage classification or the new detailed necrotico-inflammatory activity grade classification, based on the discretization of the score results of a reference population into percentiles.

According to another embodiment, the device is a meter reflecting the detailed classification, such as, for example, the new detailed fibrosis stage classification or the new detailed necrotico-inflammatory activity grade classification, based on the combination of reliable diagnostic intervals.

The present invention also relates to method for implementing an adapted patient care for an individual suffering from a liver lesion, preferably liver fibrosis or cirrhosis, comprising:

determining in the individual the presence and severity of a liver lesion, preferably liver fibrosis or cirrhosis, by:
(a) carrying out at least one non-invasive test resulting in a value;
(b) positioning the at least one test value in a class of a detailed classification of fibrosis stages or of necrotico-inflammatory activity grades based on population percentiles, wherein the detailed classification is obtained by:
   carrying out at least one non-invasive test resulting in at least one value for each subject of a reference population;
   classifying the subjects of the reference population into percentiles according to the test value obtained for said non-invasive test;
   determining for each percentile of subjects of the reference population the associated fibrosis stage(s) or necrotico-inflammatory activity grade(s) according to a fixed minimal correct classification rate and a maximal number of fibrosis stage(s) or necrotico-inflammatory activity grade(s), thus allowing the grouping of stages or grades into new classes;
(c) assessing the presence and severity of a liver lesion, preferably liver fibrosis or cirrhosis, based on the class wherein said test value, has been positioned in step (b), and
implementing an adapted patient care for the individual depending on the severity of the liver lesion, preferably liver fibrosis or cirrhosis.

According to an embodiment, the detailed classification is a detailed fibrosis classification wherein each class corresponds to less than or equal to 2 pathological fibrosis stages with reference either to the Metavir system or to the NASH-CRN scoring system or the detailed classification is a detailed necrotico-inflammatory activity classification wherein each class corresponds to less than or equal to 2 pathological activity grades.

According to an embodiment, the non-invasive test comprises the measure of at least one data issued from Vibration Controlled Transient Elastography (VCTE), also known as Fibroscan. According to another embodiment, the non-invasive test comprises at least one combination score, obtained by mathematical combination of at least one biomarker, at least one clinical marker, at least one data resulting from a physical method and/or at least one score.

According to one embodiment, the combination score is a test selected from the group consisting of ELF, FibroSpect™, APRI, FIB-4, Hepascore, Fibrotest™, CirrhoMeter™ and FibroMeter™, wherein:
ELF is a blood test based on hyaluronic acid, P3P, TIMP-1 and age;
FibroSpect™ is a blood test based on hyaluronic acid, TIMP-1 and A2M;
APRI is a blood test based on platelet and AST;
FIB-4 is a blood test based on platelet, ASAT, ALT and age;
Hepascore is a blood test based on hyaluronic acid, bilirubin, alpha2-macroglobulin, GGT, age and sex
Fibrotest™ is a blood test based on alpha2-macroglobulin, haptoglobin, apolipoprotein A1, total bilirubin, GGT, age and sex
FibroMeter™ and CirrhoMeter™ each are a blood test based on alpha2-macroglobulin, hyaluronic acid, prothrombin index, platelets, ASAT, ALAT, Urea, GGT, bilirubin, ferritin, glucose, age and/or sex.

According to one embodiment, the combination score is a FibroMeter^{V3G}, i.e., a blood test based on alpha2-macroglobulin, prothrombin index, platelets, ASAT, Urea, GGT, age and sex.

According to one embodiment, the physical method is selected from the group consisting of Doppler-ultrasonography, elastometry ultrasonography, Vibration Controlled Transient Elastography (VCTE) also known as Fibroscan, Acoustic Radiation Force Impulse (ARFI), supersonic imaging, IRM, and MNR.

According to an embodiment, the detailed classification is based on the discretization of the score results of a reference population into at least 10 percentiles of 10% of the population, preferably into at least 20 percentiles of 5% of the population, more preferably into 40 percentiles of 2.5% of the population, or more percentiles.

According to an embodiment, the individual is at risk of suffering or is suffering from a condition selected from the group consisting of a liver impairment, chronic liver disease, a hepatitis viral infection, preferably a chronic hepatitis viral infection, especially an infection caused by hepatitis B, C or D virus, a hepatotoxicity, a liver cancer, a steatosis, an alcoholic liver disease (ALD), a non-alcoholic fatty liver disease (NAFLD), a non-alcoholic steatohepatitis (NASH), an autoimmune disease, a metabolic liver disease and a disease with secondary involvement of the liver. In one embodiment, the individual is at risk of suffering or is suffering from a condition selected from the group consisting of a steatosis, a non-alcoholic fatty liver disease (NAFLD), a non-alcoholic steatohepatitis (NASH), an autoimmune disease, and a metabolic liver disease.

According to an embodiment, the individual is determined to suffer from liver fibrosis at stage F≥1, with reference either to the Metavir system or to the NASH-CRN scoring system, and the adapted patient care consists in monitoring said individual by assessing the fibrosis severity at regular intervals. According to another embodiment, the individual is determined to suffer from liver fibrosis at stage F≥2, i.e., stage F2, F3 or F4, with reference either to the Metavir system or to the NASH-CRN scoring system, and the adapted patient care consists in administering without delay at least one therapeutic agent or starting a complication screening program for applying early prophylactic or curative treatment.

According to an embodiment, the at least one therapeutic agent is an antifibrotic agent selected from the group consisting of simtuzumab, GR-MD-02, stem cell transplantation (in particular MSC transplantation), Phyllanthus urinaria, Fuzheng Huayu, S-adenosyl-L-methionine, S-nitrosol-N-acetylcystein, silymarin, phosphatidylcholine, N-acetylcysteine, resveratrol, vitamin E, losartan, telmisartan, naltrexone, RF260330, sorafenib, imatinib mesylate, nilotinib, INT747, FG-3019, oltipraz, pirfenidone, halofuginone, polaorezin, gliotoxin, sulfasalazine, rimonabant and combinations thereof.

According to an embodiment, the at least one therapeutic agent is for treating the underlying cause responsible for liver fibrosis, and/or ameliorating or alleviating the symptoms or lesions associated with the underlying cause responsible for liver fibrosis, including liver fibrosis. In one embodiment, the underlying cause responsible for liver fibrosis is a viral infection and the at least one therapeutic agent is selected from the group consisting of interferon, peginterferon 2b (pegylated IFNalpha-2b), infliximab, ribavirin, boceprevir, telaprevir, simeprevir, sofosbuvir, daclatasvir, elbasvir, grazoprevir, velpatasvir, lamivudine, adefovir dipivoxil, entecavir, telbivudine, tenofovir, clevudine, ANA380, zadaxin, CMX 157, ARB-1467, ARB-1740, ALN-HBV, BB-HB-331, Lunar-HBV, ARO-HBV, Myrcludex B, GLS4, NVR 3-778, AIC 649, JNJ56136379, ABI-H0731, AB-423, REP 2139, REP 2165, GSK3228836, GSK33389404, RNaseH Inhibitor, GS 4774, INO-1800, HB-110, TG1050, HepTcell, TomegaVax HBV, RG7795, SB9200, EYP001, CPI 431-32 and combinations thereof. In another embodiment, the underlying cause responsible for liver fibrosis is excessive alcohol consumption and the at least one therapeutic agent is selected from the group consisting of topiramate, disulfiram, naltrexone, acamprosate and baclofen. In yet another embodiment, the underlying cause responsible for liver fibrosis is a non-alcoholic fatty liver disease (NAFLD) and the at least one therapeutic agent is selected from the group consisting of telmisartan, orlistat, metformin, pioglitazone, atorvastatin, ezetimine, vitamin E, sylimarine, pentoxyfylline, ARBs, EPL, EPA-E, multistrain biotic (L. rhamnosus, L. bulgaricus), simtuzumab, obeticholic acid, elafibranor (GFT505), DUR-928, GR-MD, 02, aramchol, RG-125, cenicriviroc CVC and combinations thereof.

Another object of the invention is a method for implementing an adapted patient care for an individual suffering from a liver lesion, preferably liver fibrosis or cirrhosis, wherein the individual is afflicted with NAFLD, said method comprising:

determining in the individual with NAFLD the presence and severity of a liver lesion, preferably liver fibrosis or cirrhosis, by:
(a) carrying out at least one non-invasive test resulting in a value;
(b) positioning the at least one test value in a class of a detailed classification of fibrosis stages according to the NASH-CRN scoring system based on population percentiles, wherein the detailed classification is obtained by:
   carrying out at least one non-invasive test resulting in at least one value for each subject of a reference population;
   classifying the subjects of the reference population into percentiles according to the test value obtained for said non-invasive test;
   determining for each percentile of subjects of the reference population the associated fibrosis stages according to the NASH-CRN scoring system according to a fixed minimal correct classification rate and a maximal number of fibrosis stage(s) according to the NASH-CRN scoring system, thus allowing the grouping of stages or grades into new classes;
(c) assessing in the individual with NAFLD the presence and severity of a liver lesion, preferably liver fibrosis or cirrhosis, based on the class wherein said test value has been positioned in step (b), and
implementing an adapted patient care for the individual with NAFLD depending on the severity of the liver lesion, preferably liver fibrosis or cirrhosis.

According to an embodiment, the presence and severity of a liver lesion, preferably liver fibrosis or cirrhosis, in the individual with NAFLD is determined by:
(a) carrying out at least one FibroMeter^{V2G} resulting in a value;
(b) positioning the at least one test value in a class of a detailed classification of fibrosis stages according to the NASH-CRN scoring system based on population percentiles comprising 6 classes, namely F1±1, F1/2, F2/3, F3±1, F3/4, and F4;
(c) assessing in the individual with NAFLD the presence and severity of a liver lesion, preferably liver fibrosis or cirrhosis, based on the class wherein said the FibroMeter^{V2G} value has been positioned in step (b).

According to another embodiment, the presence and severity of a liver lesion, preferably liver fibrosis or cirrhosis, in the individual with NAFLD is determined by:
(a) carrying out at least one Fibroscan, also known as VCTE, resulting in a value;
(b) positioning the at least one test value in a class of a detailed classification of fibrosis stages according to the NASH-CRN scoring system based on population percentiles comprising 7 classes, namely F0/1, F1±1, F1/2, F2/3, F3±1, F3/4, and F4;
(c) assessing in the individual with NAFLD the presence and severity of a liver lesion, preferably liver fibrosis or cirrhosis, based on the class wherein said the Fibroscan value has been positioned in step (b).

### Definitions

About: Preceding a figure means plus or less 2% of the value of said figure.
Detailed classification: Classification in the equivalence of score in pathological degrees like fibrosis stages or activity grades. A classification comprising at least 3 classes, preferably at least 4 classes, more preferably at least 5 classes, and even more preferably at least 6 or 7 or 8 or more classes. In one embodiment, the detailed classification is a detailed fibrosis class classification.
Positioning a value in a class (respectively in a RDI): means scanning said class in order to get the information whether or not the searched value is present in the class (or RDI) or is enclosed in a range or interval present in the class (or RDI). In one embodiment, said positioning results in determining the class of a classification to which a subject belongs, and as a class is associated with Metavir fibrosis stages or Metavir activity grades, thus determining the Metavir stages or grades of said subject, without carrying out a biopsy.
Percentile: Corresponds to an interval in which a certain percent of observations falls. For example, when dividing a population in 10 percentiles of 10%, each percentile contains 10% of the population.
Single fibrosis test: Corresponds to already published blood fibrosis test obtained by a biomarker/clinical marker combination (Fibrotest™, FibroMeter™, Hepascore, APRI or FIB-4, for example), or by imaging data from Fibroscan™ also known as Vibration Controlled Transient Elastography (VCTE). The single fibrosis test provides a binary diagnosis of significant fibrosis (F≥2) or cirrhosis (F>4).
Combined fibrosis index: New fibrosis and/or necrotico-inflammatory activity test combining single fibrosis tests.
Reliable diagnosis interval (RDI): RDIs correspond to the intervals of fibrosis and/or necrotico-inflammatory activity test values wherein the individual diagnostic accuracy is considered sufficiently reliable for clinical practice. In one embodiment, the diagnostic accuracy is considered sufficiently reliable when said accuracy is of more than about 50%, preferably more than about 60, 70, 75, 80, 85, 90%. As used herein, the diagnostic accuracy refers to the percentage of patients with a correct diagnosis. In one embodiment, a diagnosis interval is reliable when more than about 50%, preferably more than about 60, 70, 75, 80, 85, 90% of subjects in said interval have a correct diagnosis. In one embodiment, a classification based on RDI derives from the cumulated cut-offs calculated for different binary diagnostic targets, usually significant fibrosis and cirrhosis.
Youden index: The index is defined as sensitivity+specificity−1, where sensitivity and specificity are calculated as proportions. Youden's index has minimum and maximum values of −1 and +1, respectively, with a value of +1 representing the optimal value for an algorithm.
Value: A value corresponds to the result of a non-invasive test, wherein the result is a number. In one embodiment, the value is a score result. In the present invention, a score result specifically means a result of a non-invasive test ranging from 0 to 1 as obtained by the logit function uses in the binary logistic regression.
Index: this is the result obtained by combining score results and or imaging data.
Score: this is the linear combination of several markers (x, y, . . . ) like a+bx+cy (a, b, c . . . being the coefficients). This often applies to the transformation of an unlimited score to a limited score by a mathematical function like logit function. In that case, the score result ranges from 0 to 1.
Method accuracy: the classification method increases the diagnostic precision (number of fibrosis stages per class). The global accuracy can be evaluated by the diagnostic accuracy (correct classification rate) and the ratio accuracy/precision.
Discrepancy score: the degree of discrepancy of diagnostic tests can be evaluated in different ways: mainly between themselves or compared to a reference (liver biopsy here). This degree can be evaluated as a grade (ordinal variable) or a score (continuous variable). The discrepancy grade between tests shows details, especially the grade of significant discrepancy (≥2 fibrosis stages). The discrepancy score 1 quantifies the magnitude of the error compared to the reference. This score was defined as follows: 0 for correct classification, then 1, 2, 3 or 4 as per the misclassification in Metavir fibrosis stages between the liver specimen and the fibrosis classification by the non-invasive test. For example, a patient with histological Metavir fibrosis 4 but classified as F0/1 by a blood test was scored 3. The mean score allows a comparison between blood tests. A low score means a low discrepancy degree.
Precision/accuracy ratio: To compare fibrosis classifications through a single index or ratio, both diagnostic accuracy and precision (i.e., the number of Metavir fibrosis stages included in each class of the fibrosis class classification) need to be taken into account. This invention includes a precision/accuracy ratio or index (IPA) for each diagnostic test as: the number of fibrosis stages per class (FSC) divided by the mean diagnostic accuracy per class (DAC). This ratio was adjusted on the number of classes per classification (CC) and the number of Metavir fibrosis stages (FMS). Thus, the final simplified formula was: IPA=(FSC×FMS)/(DAC×CC). The percentage DAC rate was expressed as a decimal, such as 0.85; other variables were expressed as raw numbers. IPA was calculated in each patient and thus permitted the statistical comparison of IPA between diagnostic tests. The reference (minimum and best) IPA was by arithmetic definition at 1 for Metavir staging. In the diagnostic algorithms including liver biopsy (LB), we weighted IPA as a function of the LB rate with the following formula: IPAB=IPA/(1−LB rate). IPAB may also be referred as “precision/accuracy/liver biopsy ratio”. The percentage LB rate was expressed as a decimal such as 0.20.
Low biopsy requirement means less than about 30%, preferably less than about 20%, more preferably less than about 10% of the patients are directed to a liver biopsy after the method of the invention was implemented.
Metavir: refers to a pathological semi-quantitative classification of liver fibrosis in 5 stages (F0-F4) based on a histological description of a liver tissue sample. The Metavir system also classifies necro-inflammatory activity in 4 grades (A0-A3).
NASH-CRN scoring system (NASH Clinical Research Network scoring system): refers to a classification system devoted to NAFLD (non-alcoholic fatty liver disease) and based on a morphological description in different classes either for steatosis (conventionally referred as grading) or lobular and portal inflammation or hepatocyte ballooning or fibrosis (conventionally referred as staging) (Brunt E M et al., Hepatology 2011; 53:810-20). This semi-quantitative (ordinal in statistics) system is the most recent and conventional histological classification. This system is also known as the Brunt grading/staging system. In particular, the NASH-CRN scoring system refers to a semi-quantitative classification of liver fibrosis in 5 stages (F0-F4) based on a morphological description of a liver tissue sample from a patient suffering from NAFLD.
Individual: refers to a mammal, preferably a human. In one embodiment, an individual may be a patient, i.e., a warm-blooded animal, more preferably a human, who/which is awaiting the receipt of, or is receiving, medical care or was/is/will be the subject of a medical procedure, or is monitored for the development or progression of a disease. In one embodiment, the individual is an adult (for example an individual above the age of 18). In another embodiment, the individual is a child (for example an individual below the age of 18). In one embodiment, the subject is a male. In another embodiment, the subject is a female.
Treating or treatment: refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. An individual is successfully “treated” for a liver lesion, preferably liver fibrosis or cirrhosis, if, after receiving at least one therapeutic agent according to the methods of the present invention, the individual shows observable and/or measurable reduction in or absence of one or more of the following: no further progression of the liver lesion or reduction in the extent of the liver lesion; relief to some extent; reduced morbidity and mortality, and/or improvement in quality of life issues. The above parameters for assessing successful treatment and improvement of the liver lesion are readily measurable by routine procedures familiar to a physician.
NAFLD Activity Score (NAS): refers to a system of scoring the histological features of non-alcoholic fatty liver disease (NAFLD). The NAS ranges from 0 to 8, and corresponds to the sum of the scores for steatosis, lobular inflammation and ballooning. The NAS scoring system is commonly used for the histological diagnosis of non-alcoholic steatohepatitis (NASH), defined as the presence of a score ≥1 for each of the three components of the NAS.

### DETAILED DESCRIPTION

The present invention relates to a non-invasive method for assessing the presence and/or severity of a lesion in an organ, and this method is based on new detailed stage classifications. In one embodiment, the detailed classification is a detailed fibrosis class classification. According to an embodiment, the organ is liver, and the detailed stage classification is a detailed fibrosis classification and/or a detailed necrotico-inflammatory activity classification. According to an embodiment, the method of the invention is useful for assessing the presence and/or severity of liver fibrosis or cirrhosis.

The method of the invention may include a first step where a non-invasive test or method of the prior art is carried out, in order to obtain for example at least one score result based on the measure of biomarkers and/or clinical markers, and/or at least one physical data such as for example medical imaging data, followed by a second step of positioning said score result(s) or data within a detailed classification comprising more than 2 classes, such as, for example, at least 3, 4, 5, 6, 7 or 8 or more classes.

In one embodiment of the invention, the non-invasive method for assessing the presence and/or severity of a lesion in an organ of an animal, including a human comprises the steps of:
(a) carrying out at least one non-invasive test resulting in a value, preferably in a score result;
(b) positioning the at least one value, preferably the at least one score result in a class of a detailed classification based on population percentiles, or based on the combination of at least two RDI; and
(c) assessing the presence and/or the severity of a lesion in an organ based on the class wherein said value or score result has been positioned in step (b).

In one embodiment of the invention, each class of said classification is associated with a risk of presence and/or of severity of a lesion in an organ.

In one embodiment, said detailed classification comprises more than 2 classes, such as, for example, at least 3, 4, 5, 6, 7 or 8 classes.

In one embodiment, said detailed classification is a detailed fibrosis classification. According to this embodiment, each class of the classification corresponds to particular Metavir fibrosis (F) stages, preferably less than or equal to 3 fibrosis stages, more preferably less than or equal to 2 fibrosis stages, even more preferably each class of the classification corresponds to one Metavir fibrosis stage. Therefore, according to this embodiment, the method of the invention determines the Metavir F stage of a subject with a diagnostic accuracy of more than about 60%, preferably more than about 70, 75, 80, 85, 90% with low or no requirement of carrying out a liver biopsy. Still according to this embodiment, the risk of presence and/or the severity of a lesion therefore is indicated by the Metavir F stage determined by the class of the fibrosis classification implemented according to the invention.

Alternatively, according to this embodiment, each class of the classification corresponds to particular fibrosis stages according to the NASH-CRN scoring system, preferably less than or equal to 3 fibrosis stages, more preferably less than or equal to 2 fibrosis stages, even more preferably each class of the classification corresponds to one fibrosis stage according to the NASH-CRN scoring system. Therefore, according to this embodiment, the method of the invention determines the fibrosis stage according to the NASH-CRN scoring system of a subject with a diagnostic accuracy of more than about 60%, preferably more than about 70, 75, 80, 85, 90% with low or no requirement of carrying out a liver biopsy. Still according to this embodiment, the risk of presence and/or the severity of a lesion therefore is indicated by the fibrosis stage according to the NASH-CRN scoring system determined by the class of the fibrosis classification implemented according to the invention.

In one embodiment, said detailed classification is a detailed necrotico-inflammatory activity classification. According to this embodiment, each class of the classification corresponds to particular Metavir A grades, preferably less than or equal to 3 grades, more preferably less than or equal to 2 grades, even more preferably each class of the classification corresponds to one grade. Therefore, according to this embodiment, the method of the invention determines the Metavir A grade of a subject with a diagnostic accuracy of more than 60%, preferably more than 70, 75, 80, 85, 90% and with low or no requirement of carrying out a liver biopsy. Still according to this embodiment, the risk of presence and/or the severity of a lesion therefore is indicated by the Metavir A grade determined by the method of the invention.

According to the invention, the non-invasive method of the invention has a diagnostic accuracy of more than about 60%, preferably more than about 70, 75, 80, 85, 90%, meaning that more than respectively about 60%, preferably more than about 70, 75, 80, 85, 90% of the patients received a correct diagnostic.

In one embodiment, said correct diagnostic corresponds to a correct diagnosis of the presence of a lesion in an organ.

In one embodiment, said correct diagnosis corresponds to a correct assessment of the severity of a lesion.

According to an embodiment, the biopsy requirement after the non-invasive method of the invention was implemented is lower than or equal to about 35, 30, 20, 15, 10, 5% of the classified patients. In one embodiment, there is no need for carrying out a liver biopsy after the non-invasive method of the invention was implemented.

According to an embodiment, the non-invasive method of the invention presents a discrepancy score lower than or equal to about 0.4, 0.3, 0.2, 0.15, 0.14, 0.13, 0.12, 0.11 or 0.10.

According to an embodiment, the non-invasive method of the invention presents a proportion of significant discrepancies, i.e., of discrepancies of more than 2 Metavir fibrosis stages lower than or equal to about 20%, preferably lower than or equal to about 10%, 7.5%, 5%, 2.5% or 1%. According to another embodiment, the non-invasive method of the invention presents a proportion of significant discrepancies, i.e., of discrepancies of more than 2 fibrosis stages according to the NASH-CRN scoring system lower than or equal to about 20%, preferably lower than or equal to about 10%, 7.5%, 5%, 2.5% or 1%.

According to an embodiment, the non-invasive method of the invention presents a precision/accuracy ratio (IPA) lower than or equal to 5, 4, 3, 2.5, 2, 1.5, 1. In one embodiment, the precision/accuracy ratio ranges from 1 to less than 5, preferably from 2 to 3, more preferably from 2.2 to 2.7, and even more preferably from 2.3 to 2.5.

According to an embodiment, the non-invasive method of the invention presents a precision/accuracy/liver biopsy ratio (IPAB) lower than or equal to 7, 6, 5, 4 or 3.

In one embodiment, the step of positioning the at least one score result or data in a class of a detailed classification is carried out using a computer.

### [Scores]

According to an embodiment, the non-invasive test comprises the measure of at least one combination score, obtained by synchronous binary combination of at least one biomarker, at least one clinical marker, at least one data resulting from a physical method and/or at least one score result. Scores that may be used for assessing presence or severity of a disease, such as, for example, a liver disease, are well known in the art. Examples of scores include, but are not limited to ELF, FIBROSpect™, APRI, FIB-4, Hepascore, Fibrotest™, FibroMeter™ and CirrhoMeter™.

ELF is a blood test based on hyaluronic acid, P3P, TIMP-1 and age.

FIBROSpect™ is a blood test based on hyaluronic acid, TIMP-1 and A2M.

APRI is a blood test based on platelet and AST.

FIB-4 is a blood test based on platelet, ASAT, ALT and age.

HEPASCORE is a blood test based on hyaluronic acid, bilirubin, alpha2-macroglobulin, GGT, age and sex.

FIBROTEST™ is a blood test based on alpha2-macroglobulin, haptoglobin, apolipoprotein A1, total bilirubin, GGT, age and sex.

FIBROMETER™ and CIRRHOMETER™ is a family of blood tests, the content of which depends on the cause of chronic liver disease and the diagnostic target with details in the following table:

| | CirrhoMeter | | | | | | | | | | | | |
|---|---|---|---|---|---|---|---|---|---|---|---|---|---|
| FibroMeter | Age | Sex | A2M | AH | PI | PLT | AST | Urea | GGT | Bili | ALT | Fer | Glu |
| F virus | X | X | X | X | X | X | X | X | | | | | |
| AOF virus | | | X | X | | X | | X | X | X | | | |
| F alcohol | X | | X | X | X | | | | | | | | |
| AOF alcohol | | | X | X | X | X | | | | | | | |
| F NAFLD | X | | | | | X | X | | X | X | X | X | X |
| AOF NAFLD | | | | X | X | X | X | | | | X | | X |

A2M: alpha2-macroglobulin,
HA: hyaluronic acid,
PI: prothrombin index,
PLT: platelets,
Bili: bilirubin,
Fer: ferritin,
Glu: glucose,
F: fibrosis stage (Metavir),
AOF: area of fibrosis,
NAFLD: non-alcoholic fatty liver disease

According to an embodiment, said score is a FibroMeter™, preferably a FibroMeter™ of second generation (FibroMeter^{2G} or FibroMeter^{V2G})

| | Age | Sex | A2M | PI | PLT | AST | Urea | HA |
|---|---|---|---|---|---|---|---|---|
| FibroMeter^{2G} | X | X | X | X | X | X | X | X |

According to an embodiment, said score is a FibroMeter™, preferably a FibroMeter™ of third generation (FibroMeter^{3G} or FibroMeter^{V3G})

| | Age | Sex | A2M | PI | PLT | AST | Urea | GGT |
|---|---|---|---|---|---|---|---|---|
| FibroMeter^{3G} | X | X | X | X | X | X | X | X |

In one embodiment, biomarkers combined in the tests of the FIBROMETER™ and the CIRRHOMETER™ family are used as single biomarkers, e.g., A2M, HA or GGT, PI, PLT, AST, urea, or as arithmetic combinations of biomarkers, such as, for example, ratios: AST/PLT or AST/ALT.

### [Biomarkers and Clinical Markers]

According to an embodiment, the at least one biomarker is selected from the group comprising Glycemia, AST (aspartate aminotransferase), ALT (alanine aminotransferase), AST/ALT, AST.ALT, Ferritin, Platelets (PLT), Prothrombin time (PT), Hyaluronic acid (HA or hyaluronate), Hemoglobin, Triglycerides, Alpha-2 macroglobulin (A2M), Platelets, Gamma-glutamyl transpeptidase (GGT), Prothrombin index (PI), Urea, Bilirubin, apolipoprotein A1 (ApoA1), type III procollagen N-terminal propeptide (P3P), gamma-globulins (GLB), sodium (NA), albumin (ALB), alkaline phosphatases (ALP), YKL-40 (human cartilage glycoprotein 39), tissue inhibitor of matrix metalloproteinase 1 (TIMP-1), cytokeratin 18 and matrix metalloproteinase 2 (MMP-2) to 9 (MMP-9).

According to an embodiment, the at least one clinical marker is selected from the group comprising weight, body mass index, age, sex, hip perimeter, abdominal perimeter and the ratio thereof, such as for example hip perimeter/abdominal perimeter.

### [Physical Data]

According to an embodiment, the non-invasive test comprises the measure of at least one data issued from a physical method of diagnosing liver fibrosis.

According to an embodiment, said physical method is selected from the group comprising medical imaging data.

According to an embodiment, the physical method is selected from the group comprising ultrasonography, especially Doppler-ultrasonography and elastometry ultrasonography and velocimetry ultrasonography (Fibroscan, Acoustic Radiation Force Impulse (ARFI), VTE, supersonic imaging), IRM, and MNR, especially MNR elastometry or velocimetry. Preferably, the data are Liver Stiffness Evaluation (LSE) data. According to a preferred embodiment of the invention, the data are issued from a Fibroscan also known as Vibration Controlled Transient Elastography (VCTE).

In one embodiment of the invention, the non-invasive test comprises the measure of at least one combination score result and of at least one data issued from a physical method of diagnosing liver fibrosis.

In one embodiment of the invention, the non-invasive test comprises carrying out a FibroMeter™, preferably a FibroMeter^{V2G}, and a Fibroscan™ (also known as VCTE).

In one embodiment of the invention, the non-invasive test comprises carrying out and mathematically combining, preferably in a binary logistic regression, a FibroMeter™, preferably a FibroMeter^{V2G}, and a Fibroscan™ (also known as VCTE).

In one embodiment of the invention, the non-invasive test comprises measuring the single markers of a FibroMeter™, preferably a FibroMeter^{V2G}, and carrying out a Fibroscan™ (also known as VCTE).

In one embodiment of the invention, the non-invasive test comprises measuring the single markers of a FibroMeter™, preferably a FibroMeter^{V2G}, carrying out a Fibroscan™ (also known as VCTE) and mathematically combining, preferably in a binary logistic regression, the obtained values.

In one embodiment of the invention, the non-invasive test comprises carrying out a CirrhoMeter™, preferably a CirrhoMeter^{V2G}, and a Fibroscan™ (also known as VCTE).

In one embodiment of the invention, the non-invasive test comprises carrying out and mathematically combining, preferably in a binary logistic regression, a CirrhoMeter™, preferably a CirrhoMeter^{V2G}, and a Fibroscan™ (also known as VCTE).

[Classifications which are Underlying the Method of the Invention]

According to the invention, the new detailed fibrosis stage and/or necrotico-inflammatory activity grade classification results from the statistical analysis of the data obtained from at least one non-invasive test as above-described, in a reference population of patients with chronic liver disease.

In one embodiment of the invention, the detailed classification of the invention is obtained by computerization of the data obtained in said reference population. In this embodiment, all the data used to make the classification are entered into a software capable of making a RDI, a combination of RDI, or percentiles.

In one embodiment of the invention the positioning of the value, preferably of the score, obtained for a subject is carried out by a computerized scan of the classification.

### Reference Population

According to an embodiment, in order to set up the new detailed fibrosis stage and/or necrotico-inflammatory activity grade classification, a reference population of patients with chronic liver disease is required. According to an embodiment, the reference population may be a population of patients affected with a Hepatitis virus, preferably with the Hepatitis C virus. According to another embodiment, the reference population may be a population affected with NAFLD (non-alcoholic fatty liver disease) and/or with NASH (non-alcoholic steatohepatitis). According to an embodiment, the reference population contains at least about 500 patients, preferably at least about 700 patients, more preferably at least about 1000 patients.

According to an embodiment, in order to set up the new detailed fibrosis stage and/or necrotico-inflammatory activity grade classification, the following data are needed for each patient of the reference population:
at least one value, which may be a non-invasive score result or an imaging data as hereinabove described, and
a histological staging, preferably a histological staging according either to the Metavir system or to the NASH-CRN scoring system, obtained by a liver biopsy.

The classifications underlying the method of the invention are selected from the group consisting of percentiles and RDI combination.

### Percentiles

This invention also relates to a percentile-based detailed fibrosis classification based on percentiles, for use in to a non-invasive method for assessing the presence and/or the severity of liver fibrosis or cirrhosis.

Percentile-based detailed fibrosis classification was elaborated as follows: the test values were segmented according to patient percentiles. They were then grouped in different classes to obtain a probability ≥75% for ≤3 fibrosis stages per class. The new fibrosis classes were called Fx, where F is the fibrosis stage(s) of the diagnostic test and x is the figures of the ≤3 fibrosis stages. In one embodiment, the fibrosis stages correspond to Metavir stages. In another embodiment, the fibrosis stated correspond to stages according to the NASH-CRN scoring system.

This invention includes but is not limited to a percentile-based detailed fibrosis classification for Fibroscan (also known as VCTE) and/or CirrhoMeter^{2G} (also referred to as CirrhoMeter^{V2G}) or FibroMeter^{2G} (also referred to as FibroMeter^{V2G}).

This invention also relates to method for drawing a detailed classification based on percentiles according to this invention, aiming at providing a new detailed fibrosis stage classification or the new detailed necrotico-inflammatory activity grade classification, based on the discretization of the results of a non-invasive test in the reference population into percentiles. According to a preferred embodiment, the result of the non-invasive test is a score result, preferably a FibroMeter™ score result.

Advantageously, the percentiles are percentiles of patients, i.e., each percentile contains the same number of patients of the reference population. In one embodiment, the percentiles are percentiles of values obtained with the non-invasive test. In an embodiment, the percentiles are not percentiles of values obtained with the non-invasive test.

According to an embodiment, the population of patients, preferably a reference population as hereinabove described, is classified into at least 10 percentiles of 10% (deciles) of the population, preferably into at least 20 percentiles of 5% of the population, more preferably into 40 percentiles of 2.5% of the population or more. Advantageously, for classifying patients of the reference population in percentiles, the values obtained by all patients of the reference population are ranged according to their numerical value, for example in ascending order, and then the first 10% of the patients corresponds to the first percentile, etc.

According to an embodiment, for each percentile, the number of patients of the reference population diagnosed after a liver biopsy at each fibrosis stage (Metavir F stage or stage according to the NASH-CRN scoring system (F0 to F4)) and/or at each Metavir A grade (A0 to A3) is quantified. Advantageously, a table is drawn, with lines corresponding to percentiles and columns corresponding to histological fibrosis stage (Metavir F stage or stage according to the NASH-CRN scoring system) and/or Metavir A grade classification. For example, in the Table 1 below, each line represents a percentile (here is shown a classification in 10 percentiles of 10%) and each column represents a Metavir F stage. Each x_{i,j} represents the number of patients of percentile i in Metavir F stage j.

TABLE 1

| | | Metavir stage | | | | |
|---|---|---|---|---|---|---|
| | | F0 | F1 | F2 | F3 | F4 |
| Percentiles | 1 | x_{1,0} | x_{1,1} | x_{1,2} | x_{1,3} | x_{1,4} |
| | 2 | x_{2,0} | x_{2,1} | x_{2,2} | x_{2,3} | x_{2,4} |
| | 3 | x_{3,0} | x_{3,1} | x_{3,2} | x_{3,3} | x_{3,4} |
| | 4 | x_{4,0} | x_{4,1} | x_{4,2} | x_{4,3} | x_{4,4} |
| | 5 | x_{5,0} | x_{5,1} | x_{5,2} | x_{5,3} | x_{5,4} |
| | 6 | x_{6,0} | x_{6,1} | x_{6,2} | x_{6,3} | x_{6,4} |
| | 7 | x_{7,0} | x_{7,1} | x_{7,2} | x_{7,3} | x_{7,4} |
| | 8 | x_{8,0} | x_{8,1} | x_{8,2} | x_{8,3} | x_{8,4} |
| | 9 | x_{9,0} | x_{9,1} | x_{9,2} | x_{9,3} | x_{9,4} |
| | 10 | x_{10,0} | x_{10,1} | x_{10,2} | x_{10,3} | x_{10,4} |

First, for each percentile (i.e., for each line of the table drawn as described hereinabove), the most frequent histological fibrosis stage (Metavir F stage or stage according to the NASH-CRN scoring system) and/or Metavir A grade is determined. For example, in Table 1, the highest x_{i,j} of each line is determined.

Second, the minimal correct classification rate is fixed per percentile at more than about 75%, preferably of more than about 80%, more preferably of more than about 85%, even more preferably of more than about 90%. On each line, the box containing the highest number is selected; further selected is the contiguous box on same line having preferably the second highest number which, when summed with the previous highest number, is equal or higher than the above-mentioned rate; this step is recommended when the previous step provided a low figure (close to 75%, e.g., 77%). When this situation does not occur, a third contiguous box having preferably the third highest number is selected, in order to equal the above-mentioned rate; this step is recommended when the additional rate provided by the third box is close to the second box obtained in previous step (e.g., 7 and 6%, respectively). Preferably, the contiguous box is selected towards to higher fibrosis stage (Metavir F stage or stage according to the NASH-CRN scoring system) and/or Metavir A grade.

For example, in Table 2 below, on each line, the highest value is in bold and the selected boxes are bounded by heavier weight lines.

When the selected columns are the same on two contiguous lines, both lines are grouped. For example, in table two, the lines of Percentiles 1 and 2 are grouped.

All the selected boxes of a group of lines form a block. For example, in Table 2, x_{1,0}, x_{1,1}, x_{2,0} and x_{2,1} form two groups, and then both groups form a block.

Each block corresponds to a class. For example, the block formed by x_{1,0}, x_{1,1}, x_{2,0} and x_{2,1} corresponds to the F0/F1 class.

For example, in Tables 1 and 2, the classification thus comprises 4 classes (F0/1, F1/2, F2/3/4 and F3/4).

In one embodiment of the invention, each class corresponds to 3, preferably 2, more preferably 1 fibrosis stage(s) (Metavir F stage(s) or stage(s) according to the NASH-CRN scoring system) or Metavir A grade(s).

The limits of the first class are determined by the lowest and highest score values obtained by a patient of said class. The upper limit of the following classes is determined by the highest score value obtained by a patient of each class. For example, in Table 2, the highest value of class F1/2 corresponds to the highest value obtained to the non-invasive test by a patient of the class F1/2 (i.e., a patient of x_{3,1}, x_{3,2}, x_{4,1}, x_{4,2}, x_{5,1} and x_{5,2}).

The method for drawing a detailed classification based on percentiles according to the invention may thus be summarized as follows:
carrying out at least one non-invasive diagnostic test resulting in at least one value, preferably at least one score result or data, for each subject of a reference population;
classifying the subjects of the reference population into percentiles, according to the value obtained for said non-invasive test, or, in other words dividing the test values of the reference population subjects into percentiles;
determining for each percentile of subjects of the reference population the associated fibrosis stage(s) or necrotico-inflammatory activity grade(s), i.e., the associated fibrosis Metavir F stage(s), fibrosis stage(s) according to the NASH-CRN scoring system or Metavir A grade(s), according to a fixed minimal correct classification rate and a maximal number of fibrosis stage(s) or necrotico-inflammatory activity grade(s), thus allowing the grouping of stages or grades into new classes of lesions. Correctly or well classified patients are true results.

In one embodiment, the maximal number of fibrosis stage(s) or necrotico-inflammatory activity grade(s) is 3. In another embodiment, the maximal number of fibrosis stage(s) or necrotico-inflammatory activity grade(s) is 2. In another embodiment, the maximal number of fibrosis stage(s) or necrotico-inflammatory activity grade(s) is 1.

According to the invention, the detailed classification may comprise classes corresponding to a grouping of a different maximal number of fibrosis stage(s) or necrotico-inflammatory activity grade(s). For example, a detailed classification obtained according to the invention may comprise a first class corresponding to the grouping of 2 fibrosis stages, a second class corresponding to 1 fibrosis stage, a third class corresponding to the grouping of 3 fibrosis stages . . . . Thus, in one embodiment, the detailed classification obtained according to the invention comprises classes corresponding to a grouping of a different maximal number of fibrosis stage(s) or necrotico-inflammatory activity grade(s).

In an embodiment, the reference population is a population of patients affected with Hepatitis C Virus, the non-invasive test is a FibroMeter™ and the population is segmented in 40 percentiles of 2.5%. In an embodiment, the reference population is a population of patients affected with Hepatitis C Virus, the non-invasive test is a Fibroscan, also known as VCTE, and the population is segmented in 40 percentiles of 2.5%. In one embodiment, the reference population is a population of patients affected with NAFLD, the non-invasive test is a FibroMeter™ and the population is segmented in 40 percentiles of 2.5%. In one embodiment, the reference population is a population of patients affected with NAFLD, the non-invasive test is a Fibroscan, also known as VCTE, and the population is segmented in 40 percentiles of 2.5%.

In another embodiment, the reference population is a population of patients affected with Hepatitis C Virus, the non-invasive test is a FibroMeter™ and the population is segmented in 20 percentiles of 5%. In another embodiment, the reference population is a population of patients affected with Hepatitis C Virus, the non-invasive test is a Fibroscan, also known as VCTE, and the population is segmented in 20 percentiles of 5%. In another embodiment, the reference population is a population of patients affected with NAFLD, the non-invasive test is a FibroMeter™ and the population is segmented in 20 percentiles of 5%. In another embodiment, the reference population is a population of patients affected with NAFLD, the non-invasive test is a Fibroscan, also known as VCTE, and the population is segmented in 20 percentiles of 5%.

According to an embodiment, the classification based on percentiles as hereinabove described comprises at least 3 classes, preferably at least 4 classes, more preferably at least 5 classes, even more preferably at least 6 classes, even more preferably at least 7 classes.

According to an embodiment, the classification based on percentiles comprises 6 classes, namely F1±1, F1/2, F2/3, F3±1, F3/4, and F4.

According to an embodiment, the classification based on percentiles comprises 7 classes, namely F0/1, F1±1, F1/2, F2/3, F3±1, F3/4, and F4.

According to an embodiment, the classification based on percentiles comprises 7 classes, namely F0/1, F1, F1/2, F1/2/3, F2/3, F2/3/4 and F3/4. According to this embodiment, the classification was implemented from a reference population to which a score based on a binary regression logistic function was performed, preferably FibroMeter™. The threshold value of each class is indicated in the second line of the table below:

| | F0/1 | F1 | F1/2 | F1/2/3 | F2/3 | F2/3/4 | F3/4 |
|---|---|---|---|---|---|---|---|
| 0 | 0.10-0.15 | 0.15-0.20 | 0.20-0.65 | 0.65-0.80 | 0.80-0.95 | 0.95-1 | 1 |
| | pref 0.14 | pref 0.17 | pref 0.56 | pref 0.72 | pref 0.86 | pref 0.97 | |

Pref: preferably

According to an embodiment, the classification based on percentiles as described hereinabove comprises at least 6 classes of fibrosis Metavir F stages. In one embodiment, the classification based on percentiles comprises 6 classes of fibrosis Metavir F stages, namely F0/1, F1/2, F2±1, F3±1, F3/4, and F4.

In one embodiment, the classification based on percentiles is constructed with test values obtained by a CirrhoMeter^{V2G} and comprises 6 classes of fibrosis Metavir F stages, namely F0/1, F1/2, F2±1, F3±1, F3/4, and F4. In another embodiment, the classification based on percentiles is constructed with test values obtained by a Fibroscan (also known as VCTE) and comprises 6 classes of fibrosis Metavir F stages, namely F0/1, F1/2, F2±1, F3±1, F3/4, and F4.

According to an embodiment, the classification based on percentiles as described hereinabove comprises at least 6 classes of fibrosis stages according to the NASH-CRN scoring system. In one embodiment, the classification based on comprises 6 classes of fibrosis stages according to the NASH-CRN scoring system, namely F1±1, F1/2, F2/3, F3±1, F3/4, and F4. In another embodiment, the classification based on percentiles comprises 7 classes of fibrosis stages according to the NASH-CRN scoring system, namely F0/1, F1±1, F1/2, F2/3, F3±1, F3/4, and F4.

In one embodiment, the classification based on percentiles is constructed with test values obtained by a FibroMeter^{V2G} and comprises 6 classes of fibrosis stages according to the NASH-CRN scoring system, namely F1±1, F1/2, F2/3, F3±1, F3/4, and F4. In another embodiment, the classification based on percentiles is constructed with test values obtained by a Fibroscan (also known as VCTE) and comprises 7 classes of fibrosis stages according to the NASH-CRN scoring system, namely F0/1, F1±1, F1/2, F2/3, F3±1, F3/4, and F4.

An example of a classification based on percentiles as hereinabove described is shown in FIG. 1.

### [Meter]

Another object of the invention is a device carrying out the method of the invention. Preferably, the device is a meter, reflecting the detailed classification, such as, for example, the new detailed fibrosis stage and/or necrotico-inflammatory activity grade classification based on percentiles as hereinabove described. Examples of meters are represented in FIG. 2A for a classification based on a FibroMeter™ score, referring to fibrosis Metavir stages (F) and to necrotico-inflammatory activity Metavir grades (A).

According to an embodiment, the meter indicates the sectors corresponding to the blocks of the classification, and the corresponding stage of fibrosis and/or grade of necrotico-inflammatory activity. The meter comprises scale marks corresponding to the score. According to an embodiment, said scale marks range from 0 to 1. The sectors are sequentially positioned on the meter. According to an embodiment, each sector of the meter has a different color.

According to a first embodiment, said meter is in the form of a line. According to a second embodiment, said meter is in the form of a disk.

According to an embodiment, the meter comprises a means, for example a line or an arrow, indicating the score result obtained by one patient to the non-invasive test and ranging the patient in a class. This indicator allows a direct visualization of the method of the invention.

### [Associated Method]

An object of this invention is thus a non-invasive method implementing the above-described classification.

In a first step of the method, a non-invasive test is carried out in a patient and gives a result in the form of a value, preferably of a score result. According to the invention, said non-invasive test is the same as the one used in the classification.

In a second step, said value, preferably said score result, is positioned on said classification, optionally on said meter, in order to range the patient in a given class.

In one embodiment, the non-invasive method for assessing the presence and/or severity of a lesion in an organ of an animal, including a human thus comprises the steps of:
(a) carrying out at least one non-invasive test resulting in a value, preferably a score result;
(b) positioning the at least one value, preferably the at least one score result in a class of a detailed classification based on population percentiles; and
(c) assessing the presence and/or the severity of a lesion in an organ based on the class wherein said score result has been positioned in step (b).

In one embodiment, the present invention relates to a method for assessing the presence and/or severity of a liver lesion, preferably liver fibrosis or cirrhosis, in an individual, comprising:
(a) carrying out, for the individual, at least one non-invasive test resulting in a value;
(b) positioning the at least one test value in a class of a detailed classification of fibrosis stages or of necrotico-inflammatory activity grades based on population percentiles, wherein the detailed classification is obtained by:
   carrying out at least one non-invasive test resulting in at least one value for each subject of a reference population;
   classifying the subjects of the reference population into percentiles according to the test value obtained for said non-invasive test, or, in other words, dividing the test values of the reference population subjects into percentiles;
   determining for each percentile of subjects of the reference population the associated fibrosis stage(s) or necrotico-inflammatory activity grade(s) according to a fixed minimal correct classification rate and a maximal number of fibrosis stage(s) or necrotico-inflammatory activity grade(s), thus allowing the grouping of stages or grades into new classes of lesions;
(c) assessing the presence and/or severity of a liver lesion, preferably liver fibrosis or cirrhosis, in the individual based on the class wherein the test value has been positioned in step (b).

According to an embodiment, the non-invasive test carried out in each subject of a reference population at step (b) is the same non-invasive test carried in the individual at step (a).

According to a preferred embodiment, the non-invasive test of step (a) and step (b) is a non-invasive fibrosis test.

In one embodiment of the invention, each class of said classification is associated with a risk of presence and/or of severity of a lesion in an organ. In one embodiment, said risk corresponds to Metavir F stages, preferably less than 3, more preferably less than 2, even more preferably one Metavir F stage(s). In one embodiment, said risk corresponds to stages according to the NASH-CRN scoring system, preferably less than 3, more preferably less than 2, even more preferably one stage(s) according to the NASH-CRN scoring system. In one embodiment, said risk corresponds to Metavir A grades, preferably 3 or less than 3, more preferably less than 2, even more preferably one Metavir A grade(s).

The accuracy of the method of the invention (i.e., the rate of well classified patients) is of at least about 75%, preferably of at least about 80%, more preferably of at least about 85%, even more preferably of at least about 90%. According to the invention, this accuracy depends on the minimal correct classification rate as described in the second step of the method for drawing the classification based on percentiles.

### RDI

### [Measure of RDI]

According to an embodiment, the new detailed fibrosis stage and/or necrotico-inflammatory activity grade classification is based on the measure of Reliable Diagnosis Intervals (RDIs) obtained from values, preferably score results of a reference population. According to a preferred embodiment, the values are score results obtained from the group comprising Fibroscan™, Fibrotest, FibroMeter™, CirrhoMeter™, Hepascore, FIB-4 and APRI.

By nature, RDIs are specific for a diagnostic target. According to a first embodiment, the diagnostic target is clinically significant fibrosis (CSF), i.e., Metavir F≥2. According to a second embodiment, the diagnostic target is severe fibrosis (SF), i.e., Metavir F≥3. According to a third embodiment, the diagnostic target is cirrhosis (C), i.e., Metavir F=4 or F=4 according to the NASH-CRN scoring system. According to a fourth embodiment, the diagnostic target is advanced fibrosis, i.e., F≥3 according to the NASH-CRN scoring system. According to a fifth embodiment, the diagnostic target is fibrotic NASH characterized by fibrosis stage F≥2 according to the NASH-CRN scoring system.

RDI measurement is well known in the art. Briefly, said measurement is based on the division of the values, preferably of the score results obtained in a reference population (as hereinabove described) into several consecutive intervals.

According to an embodiment, the measurement of the RDI comprises two, and optionally three steps:
in a first step, negative (NPV) and positive (PPV) predictive values are calculated. The method for calculating NPV and PPV from a population is well known in the art. In order to calculate these predictive values, a threshold is arbitrarily fixed. According to an embodiment, the threshold is equal to about 75%, preferably to about 80%, about 85%, about 90%, about 95%, and more preferably to about 98%;
in a second step, using NPV and PPV, two intervals are defined among the values, preferably among the score results: (i) a lower interval, defined by a value, preferably a score result inferior or equal to NPV value (score result threshold), wherein patients have more than 90% chances of not entering into the diagnostic target; and (ii) a higher interval, defined by a value, preferably a score result superior or equal to PPV value (score result threshold), wherein patients present a risk superior to 90% of entering into the diagnostic target; and
optionally, in a third step, the remaining intermediate interval (values, preferably score results, between NPV and PPV score result threshold) is segmented in two supplemental intervals according to the fibrosis and/or necrotico-inflammatory activity test value providing the diagnostic cut-off of a binary diagnosis for the diagnostic target like the maximum Youden index or the maximum diagnostic accuracy. By nature, these two intervals correspond to a class of fibrosis stages or grades different from the initial diagnostic target but with a combined prevalence of fibrosis stages and/or necrotico-inflammatory activity grades providing a class accuracy superior or equal to the predetermined threshold of fibrosis stage prevalence (e.g., ≥75%).

Therefore, for each diagnostic target, three or four intervals are defined. Each interval corresponds to a class of fibrosis stage(s) and/or necrotico-inflammatory activity grade(s). A given patient may be ranged in one of these intervals according to the score result or data obtained by said patient to the non-invasive test.

### [New Combined Fibrosis Indexes]

According to an embodiment, in order to improve the diagnostic accuracy of the method of the invention based on the RDI method as hereinabove described, single fibrosis and/or necrotico-inflammatory activity tests are combined and new combined fibrosis and/or necrotico-inflammatory activity indexes are obtained. According to an embodiment, single fibrosis and/or necrotico-inflammatory activity tests are selected from the group comprising Fibroscan™ (also known as VCTE), Fibrotest, FibroMeter™, CirrhoMeter™, Hepascore, FIB-4 and APRI.

To identify the best combination of single fibrosis and/or necrotico-inflammatory activity tests for the assessment of the presence of significant fibrosis, a stepwise binary logistic regression is performed and repeated on about 500, preferably on about 750, more preferably on about 1,000 bootstrap samples in an exploratory set of patients. The bootstrap method consists of a repeated sampling (with replacement) from the original entire dataset, followed by a stepwise logistic regression procedure in each subsample. The most frequently (>50%) selected single fibrosis and/or necrotico-inflammatory activity tests among the about 500, preferably on about 750, more preferably on about 1,000 analyses are then included in a single binary logistic regression performed in the whole population of the exploratory set.

According to an embodiment, using the regression score of such a multivariate analysis, new combined fibrosis and/or necrotico-inflammatory activity indexes are constructed for each diagnostic target, ranging from 0 to 1. According to an embodiment, for clinically significant fibrosis, said index is called “CSF-index”. According to another embodiment, a “SF-index” is constructed for the assessment of the presence of severe fibrosis as well as a “C-index” for the assessment of the presence of cirrhosis, according to methods well-known from the skilled artisan.

According to an embodiment, the combined fibrosis index used in the present invention is based on the combination of FibroMeter™, preferably FibroMeter^{V2G} and FibroScan™. According to an embodiment, the combined fibrosis index used in the present invention is based on the combination of CirrhoMeter™, preferably CirrhoMeter^{V2G} and FibroScan™.

According to an embodiment, RDIs are calculated for each of the combined fibrosis index (CSF-index, SF-index and C-index). As described hereinabove, 3 or 4 RDIs are obtained for each index.

According to an embodiment, said indexes are based on the combination of FibroMeter™ and FibroScan™ score result or data, and range between 0 and 1.

According to an embodiment, for the SCF-index, 4 intervals are defined, corresponding to the following stage of fibrosis classes: F0/1, F1/2, F1/2/3, F2/3/4. The intervals correspond to the following values of the SCF-index:
F0/1: from 0 to about 0.2 to 0.3, preferably to about 0.235;
F1/2: from 0.2 to 0.3, preferably from about 0.235; to about 0.35 to 0.45, preferably to about 0.415;
F1/2/3: from 0.35 to 0.45, preferably from about 0.415; to about 0.55 to 0.75, preferably to about 0.636;
F2/3/4: from about 0.55 to 0.75, preferably from about 0.636, to 1.

According to an embodiment, for the SF-index, 4 intervals are defined, corresponding to the following stages of fibrosis classes: F0/1/2, F1/2/3, F2/3/4, F3/4. The intervals correspond to the following values of the SF-index:
F0/1/2: from 0 to about 0.15 to 0.30, preferably to about 0.220;
F1/2/3: from 0.15 to 0.30, preferably from about 0.220; to about 0.30 to 0.45, preferably to about 0.364;
F2/3/4: from 0.30 to 0.45, preferably from about 0.364; to about 0.70 to 0.95, preferably to about 0.870;
F3/4: from about 0.70 to 0.95, preferably from about 0.870, to 1.

According to an embodiment, for the C-index, 3 intervals are defined, corresponding to the following stages of fibrosis classes: F0/1/2/3, F2/3/4, F4. The intervals correspond to the following values of the SF-index:
F0/1/2/3: from 0 to about 0.15 to 0.35, preferably to about 0.244;
F2/3/4: from 0.15 to 0.35, preferably from about 0.244; to about 0.60 to 0.95, preferably to about 0.896;
F4: from about 0.60 to 0.95, preferably from about 0.896, to 1.

In order to draw a new detailed classification based on the combination of RDIs, at least two values, preferably score results obtained by at least two non-invasive tests are measured in a reference population as hereinabove described, and at least two RDIs corresponding to said at least two values or score results are determined as hereinabove described.

In one embodiment, at least two indexes are measured as hereinabove described and at least two RDIs corresponding to said at least two indexes are determined as hereinabove described.

In order to draw a new detailed classification, RDIs obtained for each value or score result or for each index are combined. In one embodiment, said combination is carried out using a double-entry table (which may also be referred as matrix table). In one embodiment, said combination is computerized.

In one embodiment, RDIs obtained for two values, preferably score results, are combined. In one embodiment, RDIs obtained for two indexes are combined. In one embodiment, RDIs obtained for a value, preferably a score result, and RDIs obtained for an index, are combined.

In order to illustrate said combination, an example of combination using a double-entry table is shown below. When computerized, the combination of RDIs is done using the same steps.

In a first step, a double-entry table may thus be drawn, with columns corresponding to a first value (preferably score result) or index, and wherein one column corresponds to one RDI for said first value, score result or index (for example, 4 RDIs: W, X, Y and Z in Table 3). Accordingly, lines of said table corresponds to a second value (preferably score result) or index, wherein each line corresponds to one RDI for said second value, score result or index (for example, 3 RDIs A, B, and C in Table 3).

As hereinabove described, as 3 or 4 RDIs may be obtained for each value, score result or index, the table comprises 3 or 4 lines and 3 or 4 columns Therefore, the double-entry table may comprise 9, 12 or 16 cells.

TABLE 3

| | | RDI of first score result, value or index | | | |
|---|---|---|---|---|---|
| | | RDI W | RDI X | RDI Y | RDI Z |
| RDI of second | RDI A | Class AW | Class AX | Class AY | Class AZ |
| value, score | RDI B | Class BW | Class BX | Class BY | Class BZ |
| result or index | RDI C | Class CW | Class CX | Class CY | Class CZ |

In a second step, each subject of the reference population is ranged in a class, wherein a class corresponds to a cell of the double-entry table. For example, a subject ranged in the RDI W of the first score result, value or index and in the RDI B of the second score result, value or index will be ranged in the Class BW of Table 3.

In a third step, for each class (i.e., for each cell of the double-entry table), the number of patients of the reference population diagnosed after a liver biopsy at each fibrosis stage (Metavir stages or stages according to the NASH-CRN scoring system (F0 to F4)) and/or at each Metavir A grade is quantified.

Then, the most frequent histological fibrosis stage (Metavir F stage or stage according to the NASH-CRN scoring system) and/or Metavir A grade is determined for each class.

In a fourth step, the minimal classification rate is fixed per class. In one embodiment, said minimal classification rate is fixed at more than about 75%, preferably of more than about 80%, 85%, 90%.

Then, for each class, if the number of patients diagnosed with the most frequent fibrosis stage (Metavir F stage or stage according to the NASH-CRN scoring system) and/or Metavir A grade is equal or superior to the minimal classification rate fixed in the fourth step, then the class is deemed to correspond to said fibrosis stage (Metavir F stage or stage according to the NASH-CRN scoring system) and/or Metavir A grade.

For example, in Table 3, if the minimal classification rate is fixed at 75% and if more than 75% of patients of class BW have been diagnosed with F3 Metavir stage, then class BW will correspond to F3 Metavir stage, i.e., that a patient ranged in the class BW will be diagnosed with F3 Metavir stage.

When this situation does not occur, further selected is another fibrosis stage (Metavir F stage or stage according to the NASH-CRN scoring system) and/or Metavir A grade which is adjacent to the most frequent one, preferably the fibrosis stage (Metavir F stage or stage according to the NASH-CRN scoring system) and/or Metavir A grade being the second more frequent in said class.

When the number of patients diagnosed with one or the other of both selected fibrosis stages (Metavir F stages or stages according to the NASH-CRN scoring system) and/or Metavir A grades is equal or superior to the minimal classification rate fixed in the fourth step, then the class is deemed to correspond to said two fibrosis stages (Metavir F stages or stages according to the NASH-CRN scoring system) and/or Metavir A grades.

For example, in Table 3, if the minimal classification rate is fixed at 75% and if more than 75% of patients of class BW have been diagnosed with F3 or F2 Metavir stages, provided each F2 or F3 stage had less than 75% frequency, then class BW will correspond to F2/3 Metavir stage, i.e., that a patient ranged in the class BW will be diagnosed with F2/3 Metavir stage. F2/3 means F2 or F3.

When this situation does not occur, this step is repeated and another fibrosis stage (Metavir F stage or stage according to the NASH-CRN scoring system) and/or Metavir A grade which is adjacent to at least one of the most frequent one is selected, preferably the fibrosis stage (Metavir F stage or stage according to the NASH-CRN scoring system) and/or Metavir A grade being the third more frequent in said class.

When the number of patients diagnosed with one or the other of the three selected fibrosis stages (Metavir F stages or stages according to the NASH-CRN scoring system) and/or Metavir A grades is equal or superior to the minimal classification rate fixed in the fourth step, then the class is deemed to correspond to said three fibrosis stages (Metavir F stages or stages according to the NASH-CRN scoring system) and/or Metavir A grades.

For example, in Table 3, if the minimal classification rate is fixed at 75% and if more than 75% of patients of class BW have been diagnosed with F1, F2 or F3 Metavir stages, provided each F1, F2 or F3 stage had less than 75% frequency, then class BW will correspond to F1/2/3 Metavir stage, i.e., that a patient ranged in the class BW will be diagnosed with F1/2/3 Metavir stage.

In a fifth step, when two adjacent classes in the double-entry table have been determined to be associated with the same fibrosis stages (Metavir F stages or stage according to the NASH-CRN scoring system) and/or Metavir A grades, then both classes are grouped. For example, in Table 3, if the classes BW and BX both correspond to F2/3, then they are grouped.

In one embodiment, the classification comprises more than 3 classes, preferably 4, 5, 6, 7 or more classes.

In one embodiment, the classification comprises more classes than the number of RDIs for the first value or score result or index and/or than the number of RDIs for the second value or score result or index. Therefore, according to this embodiment, the classification based on the combination of RDIs allows a more precise classification of patients than non-combined RDIs.

In one embodiment, each class corresponds to 3, preferably 2, more preferably 1 Metavir F stage(s), stages according to the NASH-CRN scoring system or Metavir A grade(s).

The method for drawing a detailed classification based on the combination of RDIs according to the invention may thus be summarized as follows:
carrying out at least two non-invasive tests resulting in at least two values, preferably score results for each patient of a reference population;
optionally combining said values or score results in a mathematical function in order to obtain at least two indexes;
determining for each value, score result or index the RDIs;
combining the RDIs, using a double-entry table or by computerization, thereby determining classes;
determining for each class the associated fibrosis stage(s) or necrotico-inflammatory activity grade(s), i.e., the associated fibrosis Metavir F stage(s), fibrosis stage(s) according to the NASH-CRN scoring system or Metavir A grade(s), according to a minimal correct classification rate to be fixed.

According to an embodiment, when indexes are used, the RDIs obtained for each index are combined, in order to obtain a more detailed classification. According to a first embodiment, the RDIs of CSF-index and of SF-index are combined, leading to the “CSF/SF classification”. According to a first embodiment, the RDIs of CSF-index and of C-index are combined, leading to the “CSF/C classification”.

According to an embodiment, said combination corresponds to the drawing of a double entry table, comprising columns corresponding to RDIs of the first index, and lines corresponding to RDIs of the second index. An example of such a double entry table based on Metavir F stages, is Table 4 below.

TABLE 4

| | | RDI of CSF-Index | | | |
|---|---|---|---|---|---|
| | | F0/1 | F1/2 | F1/2/3 | F2/3/4 |
| RDI of SF-Index | F0/1/2 | F0/1 | F1/2 | F1/2 | |
| | F1/2/3 | | | F1/2/3 | F2/3 |
| | F2/3/4 | | | | F2/3/4 |
| | F3/4 | | | | F4 |

For each patient, the calculated CSF-Index is positioned in one of the RDIs of CSF-index, and the calculated SF-Index is positioned in one of the RDI of SF-Index. As an example, a patient with a CSF-Index positioned in the class F2/3/4 and with a SF-Index positioned in the class F1/2/3 may be classified in a narrower class F2/3.

Therefore, as shown in Table 4, the combination of RDIs or CSF-index and of SF-index leads to a “CSF/SF classification”, comprising 6 classes, namely F0/1, F1/2, F1/2/3, F2/3, F2/3/4 and F4.

Accordingly, and as illustrated on Table 5 below, the combination of RDIs or CSF-index and of CF-index leads to a “SCF/CF classification” comprising 7 classes, namely F0/1, F1/2, F1/2/3, F2, F2/3, F2/3/4 and F4.

TABLE 5

| | | RDI of CSF-Index | | | |
|---|---|---|---|---|---|
| | | F0/1 | F1/2 | F1/2/3 | F2/3/4 |
| RDI of CF- | F0/1/2/3 | F0/1 | F1/2 | F1/2/3 | F2/3 |
| Index | F2/3/4 | | F2 | F2/3 | F2/3/4 |
| | F4 | | | | F4 |

According to an embodiment, said new fibrosis stage and/or necrotico-inflammatory activity grade classification comprises at least 3, preferably at least 4, more preferably at least 5 classes, even more preferably at least 6 or 7 classes.

### [Meter]

Another object of the invention is a device carrying out the method of the invention. Preferably, the device is a meter, reflecting the detailed classification, such as, for example, the new detailed fibrosis stage and/or necrotico-inflammatory activity grade classification as hereinabove described. Examples of meters are represented in FIG. 2B for a fibrosis classification based on the combination of FibroMeter™ and FibroScan™ (also known as VCTE).

According to a first embodiment, said meter is in the form of a line. According to a second embodiment, said meter is in the form of a disk.

According to an embodiment, the Meter of the invention is segmented in different sectors, each sector corresponding to a class of the classification. According to an embodiment, each sector of the meter has a different color.

### [Associated Non-Invasive Method]

An object of this invention is thus a non-invasive method implementing the new detailed fibrosis stage and/or necrotico-inflammatory activity grade classification based on the combination of RDIs as hereinabove described.

In one embodiment of the invention, the non-invasive method for assessing the presence and/or severity of a lesion in an organ of an animal, including a human comprises the steps of:
1. carrying out at least two non-invasive tests resulting in at least two values preferably at least two score results; and/or at least one score result and at least one physical data; and/or at least two physical data;
2. optionally combining said at least two values or score result in a mathematical function, thereby obtaining at least two indexes;
3. positioning the at least two score results or values of step (a), or the at least two indexes of step (b) in a class of a detailed classification based on the combination of at least two RDIs; and
4. assessing the presence and/or the severity of a lesion in an organ based on the class wherein said score result has been positioned in step (c).

In one embodiment, each class of said classification is associated with a risk of presence and/or of severity of a lesion in an organ. In one embodiment, each class of said classification is associated with 3, preferably 2, more preferably 1 Metavir F stage(s). In one embodiment, each class of said classification is associated with 3, preferably 2, more preferably 1 fibrosis stage(s) according to the NASH-CRN scoring system. In one embodiment, each class of said classification is associated with 3, preferably 2, more preferably 1 Metavir A grade(s).

In one embodiment of the invention, said non-invasive tests comprise Fibroscan™, Fibrotest, FibroMeter™, CirrhoMeter™, Hepascore, FIB-4 and APRI.

According to the invention, said non-invasive tests are the same as the ones used to obtain the classification. Preferably, said non-invasive tests are Fibroscan™ and FibroMeter™.

According to an embodiment, in order to position the values, score results or index in step (c), the first value, score result or index and the second value, score result or index are ranged respectively in a RDI of the first value, score result or index and in a RDI of the second value, score result or index; and the RDIs thus obtained are then crossed together.

In one embodiment, said positioning and said crossing are obtained using a double-entry table. In another embodiment, said positioning and said crossing are computerized.

In one embodiment of the invention, the non-invasive method for assessing the presence and/or severity of a lesion in an organ of an animal, including a human thus comprises the steps of:
(a) carrying out at least two non-invasive tests resulting in at least two values, preferably at least two scores and/or at least one score result and at least one physical data, and/or at least two physical data;
(b) positioning each of the at least two score results or values in a reliable diagnostic interval (RDI); thereby obtaining at least two RDIs;
(c) crossing the at least two RDI of step (b) for a final positioning in a class; and
(d) assessing the presence and/or the severity of a lesion in an organ based on the class wherein said score result has been positioned in step (c).

In one embodiment of the invention, the method of the invention comprises the following steps:

In a first step of the method, at least two non-invasive tests are carried out in a patient and at least two values, preferably at least two score results are obtained. According to the invention, said non-invasive tests are selected from the group comprising Fibroscan™, Fibrotest, FibroMeter™, CirrhoMeter™, Hepascore, FIB-4 and APRI, and are the same as the ones used to obtain the classification. Preferably, said non-invasive tests are Fibroscan™ and FibroMeter™.

In a second step, both results are combined using three binary logistic regressions to obtain three indexes (CSF-Index, SF-Index and C-Index), ranging from 0 to 1.

In a third step, the CSF-Index SF-Index and C-Index are ranged respectively in a RDI of CSF-Index, in a RDI of SF-Index and in a RDI of C-Index.

In a fourth step, the patient is positioned in a fibrosis stage and/or necrotico-inflammatory activity grade class. In one embodiment, said positioning is obtained using a double entry table (see Tables 4 and 5). In another embodiment, said positioning is computerized.

In one embodiment of the invention, the non-invasive method for assessing the presence and/or severity of a lesion in an organ of an animal, including a human thus comprises the steps of:
(a) carrying out at least two non-invasive tests resulting in at least two score results or physical data;
(b) combining said at least two score results or physical data in at least two mathematical functions, preferably at least two binary logistic regressions, thereby obtaining at least two indexes;
(c) positioning each of the at least two indexes in a reliable diagnostic interval (RDI), thereby obtaining at least two RDI;
(d) crossing the at least two RDI of step (b) for a final positioning in a class; and
(e) assessing the presence and/or the severity of a lesion in an organ based on the class wherein said score has been positioned in step (d).

According to an embodiment, the accuracy of said non-invasive method (i.e., the rate of well classified patients) is of at least about 75%, preferably of at least about 80%, more preferably of at least about 85%, even more preferably of at least about 90%.

### [Advantages]

The non-invasive methods of the present invention, implementing new detailed fibrosis stage and/or necrotico-inflammatory activity grade classifications based on percentiles or on the combination of RDIs, both present the following advantages:
Increased precision, due to the number of classes of the classification;
Statistically significant increase in diagnostic accuracy, with an accuracy >60%;
Low discrepancy score;
The possibility to target this classification towards different diagnostic targets;
The possibility to apply this classification to different non-invasive tests or methods, especially by combining two or more non-invasive tests.
   The increase of reliability provided by the method of the invention is also shown by the improved precision/accuracy ratios, with comparison to binary diagnosis tests. Especially, the detailed classifications of the invention present better precision/accuracy ratios compared to binary diagnosis. For example, in cirrhosis, results were obtained with a detailed classification based on percentiles showing a precision/accuracy ratio from 2.3 to 2.5 in single test classifications versus more than 5 for the best binary diagnosis for cirrhosis;
   The detailed classification of the invention allows narrowing, if not erasing, the zone of the classification wherein a biopsy is required (“grey zone”). A grey zone may correspond, for example, to a class of the classification wherein the patient is classified as F1/2, i.e., may have no fibrosis (F1) or significant fibrosis (F2). Consequently, the use of the detailed classification of the invention leads to a low requirement (such as, for example, less than 30%) or no requirement of biopsy.

Therefore, the detailed classification presents two main advantages: on one hand it adds precision to accuracy, and on the other, it resolves the diagnostic uncertainty in the “grey zone” of binary diagnosis, especially for Fibroscan (also known as VCTE). Indeed, this latter, expressed in kPa, could not be interpreted in terms of diagnostic probability, contrary to most blood tests, which can be interpreted as a probability of the diagnostic target.

### [Method of Treatment]

The present invention also relates to a method for treating an individual identified as suffering from a liver lesion, such as, for example, liver fibrosis or cirrhosis. Thus, the present invention also relates to a method for implementing an adapted patient care for an individual suffering from a liver lesion, preferably liver fibrosis or cirrhosis, comprising: determining in the individual the presence and severity of a liver lesion, preferably liver fibrosis or cirrhosis, as described hereinabove by:
(a) carrying out at least one non-invasive test resulting in a value;
(b) positioning the at least one value in a class of a detailed classification; and
(c) assessing the presence and severity of a liver lesion, preferably liver fibrosis or cirrhosis, based on the class wherein said test value has been positioned in step (b), and
   implementing an adapted patient care for the individual depending on the severity of the liver lesion, preferably liver fibrosis or cirrhosis.

In one embodiment, the method of the invention for implementing an adapted patient care for an individual suffering from a liver lesion, preferably liver fibrosis or cirrhosis, comprises:

determining in the individual the presence and severity of a liver lesion, preferably liver fibrosis or cirrhosis, by:
(a) carrying out at least one non-invasive test resulting in a value;
(b) positioning the at least one value in a class of a detailed classification based on population percentiles as described hereinabove; and
(c) assessing the presence and severity of a liver lesion, preferably liver fibrosis or cirrhosis, based on the class wherein said test value has been positioned in step (b), and
   implementing an adapted patient care for the individual depending on the severity of the liver lesion, preferably liver fibrosis or cirrhosis.

In another embodiment, the method of the invention for implementing an adapted patient care for an individual suffering from a liver lesion, preferably liver fibrosis or cirrhosis, comprises:

determining in the individual the presence and severity of a liver lesion, preferably liver fibrosis or cirrhosis, by:
(a) carrying out at least one non-invasive test resulting in a value;
(b) positioning the at least one value in a class of a detailed classification based on the combination of at least two reliable diagnostic intervals (RDIs) as described hereinabove; and
(c) assessing the presence and severity of a liver lesion, preferably liver fibrosis or cirrhosis, based on the class wherein said test value has been positioned in step (b), and
   implementing an adapted patient care for the individual depending on the severity of the liver lesion, preferably liver fibrosis or cirrhosis.

The present invention also relates to a method for implementing an adapted patient care for an individual suffering from a liver lesion, preferably liver fibrosis or cirrhosis, comprising:

determining in the individual the presence and severity of a liver lesion, preferably liver fibrosis or cirrhosis, by:
(a) carrying out at least one non-invasive test resulting in a value;
(b) positioning the at least one test value in a class of a detailed classification of fibrosis stages or of necrotico-inflammatory activity grades based on population percentiles, wherein the detailed classification is obtained by:
   carrying out at least one non-invasive test resulting in at least one value for each subject of a reference population;
   classifying said subjects of the reference population into percentiles according to the test value obtained for said non-invasive test;
   determining for each percentile of subjects of the reference population the associated fibrosis stage(s) or necrotico-inflammatory activity grade(s) according to a fixed minimal correct classification rate and a maximal number of fibrosis stage(s) or necrotico-inflammatory activity grade(s), thus allowing the grouping of stages or grades into new classes;
(c) assessing the presence and severity of a liver lesion, preferably liver fibrosis or cirrhosis, based on the class wherein said test value, has been positioned in step (b), and
   implementing an adapted patient care for the individual depending on the severity of the liver lesion, preferably liver fibrosis or cirrhosis.

In one embodiment, the individual is determined to suffer from liver fibrosis at stage F1, with reference either to the Metavir system or to the NASH-CRN scoring system, and the adapted patient care consists in monitoring said individual by assessing the fibrosis severity at regular intervals. In another embodiment, the individual is determined to suffer from liver fibrosis at stage F≥1, with reference either to the Metavir system or to the NASH-CRN scoring system, and the adapted patient care consists in monitoring said individual by assessing the fibrosis severity at regular intervals.

In one embodiment, the fibrosis severity is assessed every 3 months, every 6 months, every 9 months, every 12 months, every 15 months, every 18 months, every 24 months, or every 36 months.

In one embodiment, the individual is determined to suffer from liver fibrosis at stage F≥2, with reference either to the Metavir system or to the NASH-CRN scoring system, and the adapted patient care consists in administering without delay at least one therapeutic agent or starting a complication screening program for applying early prophylactic or curative treatment.

In one embodiment, the individual is determined to suffer from severe liver fibrosis at stage F≥3, with reference either to the Metavir system or to the NASH-CRN scoring system, and the adapted patient care consists in administering without delay at least one therapeutic agent and optionally starting a complication screening program for applying early prophylactic or curative treatment.

In one embodiment, the individual is determined to suffer from cirrhosis, i.e., liver fibrosis at stage F4 (F=4) with reference either to the Metavir system or to the NASH-CRN scoring system, and the adapted patient care consists in administering without delay at least one therapeutic agent and starting a complication screening program for applying curative treatment.

In one embodiment, the individual, preferably an individual afflicted with non-alcoholic fatty liver disease (NAFLD), is determined to suffer from advanced liver fibrosis at stage F≥3 with reference to the NASH-CRN scoring system, and the adapted patient care consists in administering without delay at least one therapeutic agent and optionally starting a complication screening program for applying early prophylactic or curative treatment.

In one embodiment, the individual, preferably an individual afflicted with non-alcoholic steatohepatitis (NASH), more preferably an individual afflicted with NASH with a NAFLD Activity Score (NAS)≥4, is determined to suffer from fibrotic NASH with a fibrosis at stage F≥2 with reference to the NASH-CRN scoring system, and the adapted patient care consists in administering without delay at least one therapeutic agent and optionally starting a complication screening program for applying early prophylactic or curative treatment.

Examples of therapeutic agents include, but are not limited to, bezafibrate, S-adenosyl-L-methionine, S-nitrosol-N-acetylcystein, silymarin, phosphatidylcholine, N-acetylcysteine, resveratrol, vitamin E, pentoxyphilline (or pentoxyfilline) alone or in combination with tocopherol, pioglitazone alone or in combination with vitamin E, lovaza (fish oil), PPC alone or in combination with an antiviral therapy (e.g., IFN), INT747, peginterferon 2b (pegylated IFNalpha-2b), a combination of infliximab, and ribavirin, stem cell transplantation (in particular MSC transplantation), candesartan, losartan, telmisartan, irbesartan, ambrisentan, FG-3019, Phyllanthus urinaria, Fuzheng Huayu, warfarin, insulin, colchicine, corticosteroids, naltrexone, RF260330, sorafenib, imatinib mesylate, nilotinib, pirfenidone, halofuginone, polaorezin, gliotoxin, sulfasalazine, rimonabant, simtuzumab, GR-MD-02, boceprevir, telaprevir, simeprevir, sofosbuvir, daclatasvir, elbasvir, grazoprevir, velpatasvir, lamivudine, adefovir dipivoxil, entecavir, telbivudine, tenofovir, clevudine, ANA380, zadaxin, CMX 157, ARB-1467, ARB-1740, ALN-HBV, BB-HB-331, Lunar-HBV, ARO-HBV, Myrcludex B, GLS4, NVR 3-778, AIC 649, JNJ56136379, ABI-H0731, AB-423, REP 2139, REP 2165, GSK3228836, GSK33389404, RNaseH Inhibitor, GS 4774, INO-1800, HB-110, TG1050, HepTcell, TomegaVax HBV, RG7795, SB9200, EYP001, CPI 431-32, topiramate, disulfiram, naltrexone, acamprosate, baclofen, methadone, buprenorphine, orlistat, metformin, atorvastatin, ezetimine, ARBs, EPL, EPA-E, multistrain biotic (L. rhamnosus, L. bulgaricus), obeticholic acid, elafibranor (GFT505), DUR-928, GR-MD, 02, aramchol, RG-125, cenicriviroc CVC, rosiglitazone, MSDC-0602K, GS-9674, LJN452, LMB763, EDP-305, elafibranor, saroglitazar, IVA337, NGM282, PF-05231023, BMS-986036, aramchol, volixibat, GS-0976, liraglutide, semaglutide exenatide, taspoglutide, taurine, polyenephosphatidylcholine, MGL-3196, vitamin C, GS-4997, sitagliptin, alogliptin, vildagliptin, saxagliptin, linagliptin, PXS-4728A, VLX-103, hyperimmune bovine clostrum, nalmefene, emricasan, milk thistle; and probiotics and combinations thereof.

In one embodiment, the at least one therapeutic agent is an antifibrotic agent selected from the group consisting of simtuzumab, GR-MD-02, stem cell transplantation (in particular MSC transplantation), Phyllanthus urinaria, Fuzheng Huayu, S-adenosyl-L-methionine, S-nitrosol-N-acetylcystein, silymarin, phosphatidylcholine, N-acetylcysteine, resveratrol, vitamin E, losartan, telmisartan, naltrexone, RF260330, sorafenib, imatinib mesylate, nilotinib, INT747, FG-3019, oltipraz, pirfenidone, halofuginone, polaorezin, gliotoxin, sulfasalazine, rimonabant and combinations thereof.

In one embodiment, the at least one therapeutic agent is for treating the underlying cause responsible for liver fibrosis, and/or ameliorating or alleviating the symptoms or lesions associated with the underlying cause responsible for liver fibrosis, including liver fibrosis.

In one embodiment, the underlying cause responsible for liver fibrosis is a viral infection and the at least one therapeutic agent is selected from the group consisting of interferon, peginterferon 2b (pegylated IFNalpha-2b), infliximab, ribavirin, boceprevir, telaprevir, simeprevir, sofosbuvir, daclatasvir, elbasvir, grazoprevir, velpatasvir, lamivudine, adefovir dipivoxil, entecavir, telbivudine, tenofovir, clevudine, ANA380, zadaxin, CMX 157, ARB-1467, ARB-1740, ALN-HBV, BB-HB-331, Lunar-HBV, ARO-HBV, Myrcludex B, GLS4, NVR 3-778, AIC 649, JNJ56136379, ABI-H0731, AB-423, REP 2139, REP 2165, GSK3228836, GSK33389404, RNaseH Inhibitor, GS 4774, INO-1800, HB-110, TG1050, HepTcell, TomegaVax HBV, RG7795, SB9200, EYP001, CPI 431-32 and combinations thereof.

In one embodiment, the underlying cause responsible for liver fibrosis is excessive alcohol consumption and the at least one therapeutic agent is selected from the group consisting of topiramate, disulfiram, naltrexone, acamprosate and baclofen.

In one embodiment, the underlying cause responsible for liver fibrosis is a non-alcoholic fatty liver disease (NAFLD) and the at least one therapeutic agent is selected from the group consisting of telmisartan, orlistat, metformin, pioglitazone, atorvastatin, ezetimine, vitamin E, sylimarine, pentoxyfylline, ARBs, EPL, EPA-E, multistrain biotic (L. rhamnosus, L. bulgaricus), simtuzumab, obeticholic acid, elafibranor (GFT505), DUR-928, GR-MD, 02, aramchol, RG-125, cenicriviroc CVC and combinations thereof.

In one embodiment, the underlying cause responsible for liver fibrosis is a non-alcoholic steatohepatitis (NASH), preferably fibrotic NASH, and the at least one therapeutic agent is selected from the group consisting of insulin sensitizers (such as rosiglitazone, pioglitazone and MSDC-0602K); farnesoid X receptor (FXR) agonists (such as obeticholic acid (also referred to as OCA), GS-9674, LJN452, LMB763 and EDP-305); Peroxisome Proliferator-Activated Receptor α/δ (PPAR α/δ) agonists (such as elafibranor, saroglitazar and IVA337); fibroblast growth factor 19 (FGF19) analogs (such as NGM282); fibroblast growth factor 21 (FGF21) analogs (such as PF-05231023); recombinant FGF21 (such as BMS-986036); stearoyl-coenzyme A desaturase 1 (SCD1) inhibitors (such as aramchol); apical sodium-dependent bile acid transporter (ASBT) inhibitors (such as volixibat); acetyl-coA carboxylase (ACC) inhibitors (such as GS-0976); glucagon-like peptide-1 (GLP-1) analogs (such as liraglutide, semaglutide exenatide and taspoglutide); ursodeoxycholic acid and norursodeoxycholic acid (NorUDCA); taurine; polyenephosphatidylcholine; thyroid hormone receptor (THR) β-agonists (such as MGL-3196); antioxidant agents (such as vitamin E and vitamin C); apoptosis signal-regulating kinase 1 (ASK1) inhibitors (such as GS-4997); DPP-4 inhibitors (such as sitagliptin, alogliptin, vildagliptin, saxagliptin, and linagliptin); vascular adhesion protein-1 (VAP-1) inhibitors (such as PXS-4728A); phosphodiesterase-4 (PDE-4) inhibitors; angiotensin II-1 type receptor antagonists (such as losartan and telmisartan); anti-inflammatory compounds (such as cenicriviroc, VLX-103 (oral pentamidine) and hyperimmune bovine clostrum); Toll-like receptor 4 antagonists (such as nalmefene); caspase inhibitors (such as emricasan); pentoxifylline; S-adenosylmethionine; milk thistle; and probiotics.

In one embodiment, the treated individual is administered both at least one antifibrotic agent and at least one therapeutic agent for treating the underlying cause responsible for liver fibrosis, and/or ameliorating or alleviating the symptoms or lesions associated with the underlying cause responsible for liver fibrosis.

In one embodiment, the method for implementing an adapted patient care for an individual suffering from a liver lesion, preferably liver fibrosis or cirrhosis, comprises: determining in the individual the presence and severity of a liver lesion, preferably liver fibrosis or cirrhosis, by:
(a) carrying out at least one Fibroscan, also known as VCTE, resulting in a value;
(b) positioning the at least one test value in a class of a detailed classification of fibrosis Metavir F stages based on population percentiles comprising 6 classes, namely F0/1, F1/2, F2±1, F3±1, F3/4, and F4;
(c) assessing in the individual the presence and severity of a liver lesion, preferably liver fibrosis or cirrhosis, based on the class wherein said the Fibroscan value has been positioned in step (b), and
   implementing an adapted patient care for the individual depending on the severity of the liver lesion, preferably liver fibrosis or cirrhosis.

In another embodiment, the method for implementing an adapted patient care for an individual suffering from a liver lesion, preferably liver fibrosis or cirrhosis, comprises: determining in the individual the presence and severity of a liver lesion, preferably liver fibrosis or cirrhosis, by:
(a) carrying out at least one CirrhoMeter™, preferably a CirrhoMeter^{V2G}, resulting in a value;
(b) positioning the at least one test value in a class of a detailed classification of fibrosis Metavir F stages based on population percentiles comprising 6 classes, namely F0/1, F1/2, F2±1, F3±1, F3/4, and F4;
(c) assessing in the individual the presence and severity of a liver lesion, preferably liver fibrosis or cirrhosis, based on the class wherein said the CirrhoMeter™ value has been positioned in step (b), and
   implementing an adapted patient care for the individual depending on the severity of the liver lesion, preferably liver fibrosis or cirrhosis.

In a particular embodiment, the present invention relates to a method for implementing an adapted patient care for an individual with NAFLD suffering from a liver lesion, preferably liver fibrosis or cirrhosis, comprising:

determining in the individual with NAFLD the presence and severity of a liver lesion, preferably liver fibrosis or cirrhosis, by:
(a) carrying out at least one non-invasive test resulting in a value;
(b) positioning the at least one test value in a class of a detailed classification of fibrosis stages according to the NASH-CRN scoring system based on population percentiles, wherein the detailed classification is obtained by:
   carrying out at least one non-invasive test resulting in at least one value for each subject of a reference population;
   classifying said subjects of the reference population into percentiles according to the test value obtained for said non-invasive test;
   determining for each percentile of subjects of the reference population the associated fibrosis stages according to the NASH-CRN scoring system according to a fixed minimal correct classification rate and a maximal number of fibrosis stage(s) according to the NASH-CRN scoring system, thus allowing the grouping of stages or grades into new classes;
(c) assessing in the individual with NAFLD the presence and severity of a liver lesion, preferably liver fibrosis or cirrhosis, based on the class wherein said test value has been positioned in step (b), and
   implementing an adapted patient care for the individual with NAFLD depending on the severity of the liver lesion, preferably liver fibrosis or cirrhosis.

In one embodiment, the method for implementing an adapted patient care for an individual with NAFLD suffering from a liver lesion, preferably liver fibrosis or cirrhosis, comprises:

determining in the individual with NAFLD the presence and severity of a liver lesion, preferably liver fibrosis or cirrhosis, by:
(a) carrying out at least one FibroMeter^{V2G} resulting in a value;
(b) positioning the at least one test value in a class of a detailed classification of fibrosis stages according to the NASH-CRN scoring system based on population percentiles comprising 6 classes, namely F1±1, F1/2, F2/3, F3±1, F3/4, and F4;
(c) assessing in the individual with NAFLD the presence and severity of a liver lesion, preferably liver fibrosis or cirrhosis, based on the class wherein said the FibroMeter^{V2G} value has been positioned in step (b), and
   implementing an adapted patient care for the individual with NAFLD depending on the severity of the liver lesion, preferably liver fibrosis or cirrhosis.

In one embodiment, treatment is provided to the individual with NAFLD determined to have a fibrosis classified as F2/3, F3±1, F3/4 or F4.

In another embodiment, the method for implementing an adapted patient care for an individual with NAFLD suffering from a liver lesion, preferably liver fibrosis or cirrhosis, comprises:

determining in the individual with NAFLD the presence and severity of a liver lesion, preferably liver fibrosis or cirrhosis, by:
(a) carrying out at least one Fibroscan, also known as VCTE, resulting in a value;
(b) positioning the at least one test value in a class of a detailed classification of fibrosis stages according to the NASH-CRN scoring system based on population percentiles comprising 7 classes, namely F0/1, F1±1, F1/2, F2/3, F3±1, F3/4, and F4;
(c) assessing in the individual with NAFLD the presence and severity of a liver lesion, preferably liver fibrosis or cirrhosis, based on the class wherein said the Fibroscan value has been positioned in step (b), and
   implementing an adapted patient care for the individual with NAFLD depending on the severity of the liver lesion, preferably liver fibrosis or cirrhosis.

In one embodiment, treatment is provided to the individual with NAFLD determined to have a fibrosis classified as F2/3, F3±1, F3/4 or F4.

In one embodiment, the treatment provided to the individual with NAFLD determined to have a fibrosis classified as F2/3, F3±1, F3/4 or F4 comprises administering at least one therapeutic agent selected from the group consisting of telmisartan, orlistat, metformin, pioglitazone, atorvastatin, ezetimine, vitamin E, sylimarine, pentoxyfylline, ARBs, EPL, EPA-E, multistrain biotic (L. rhamnosus, L. bulgaricus), simtuzumab, obeticholic acid, elafibranor (GFT505), DUR-928, GR-MD, 02, aramchol, RG-125, cenicriviroc CVC and combinations thereof.

### BRIEF DESCRIPTION OF THE DRAWINGS

FIG. 1: Example of classification based on percentiles.

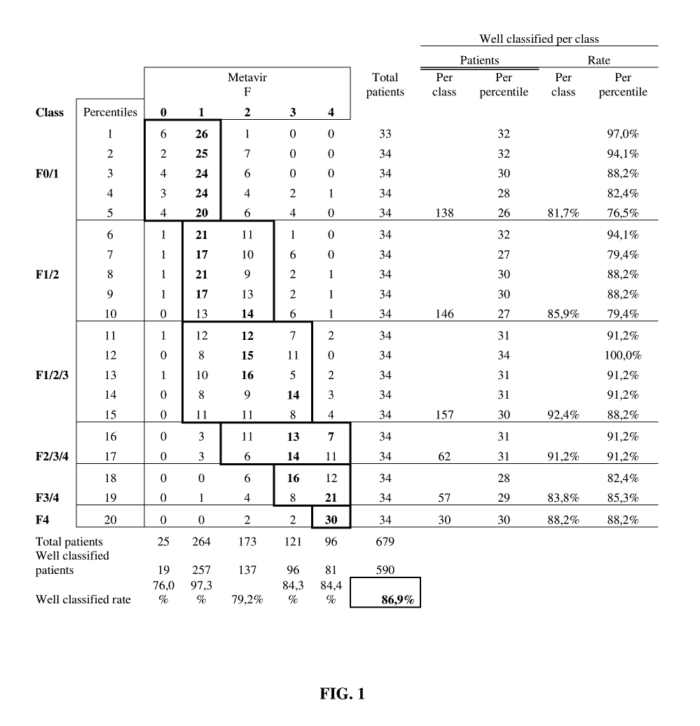

FIG. 2: Examples of Meters of the invention. A: Meter reflecting the detailed classification based on percentiles, referring to fibrosis (F) and to necrotico-inflammatory activity (A). B: Meter reflecting the detailed classification based on the combination of RDIs.

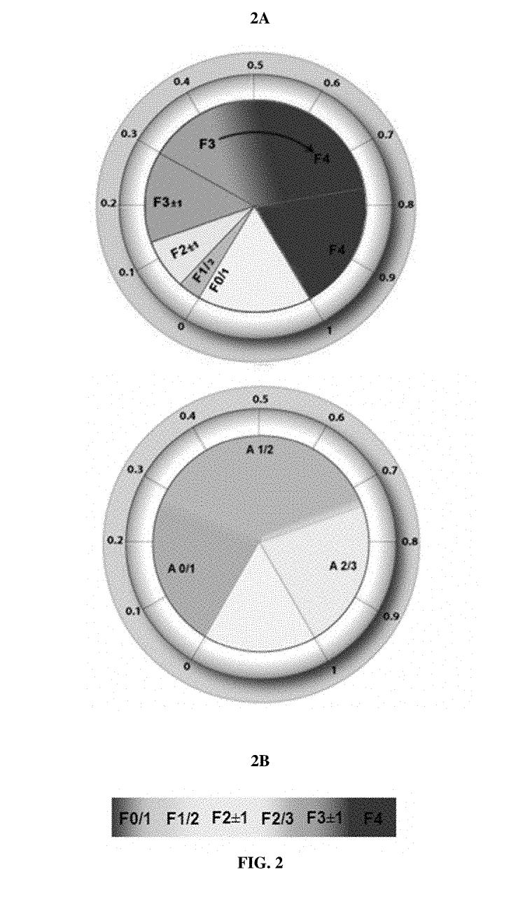

FIG. 3: Various fibrosis stage classifications. A: Histological Metavir fibrosis stages. B-E: Fibrosis stage classifications by non-invasive tests; B: Fibrotest classification; C: FibroMeter classification; D: Fibroscan classification; E: new CSF/SF classification derived from the association of new fibrosis indexes combining FibroMeter and Fibroscan.

FIG. 4: Study methodology. Implementation of new fibrosis stage classifications from new combined fibrosis indexes (exploratory set). BLR: binary logistic regression, RDI: reliable diagnosis intervals.

FIG. 5: Reliable diagnosis intervals of CSF-, SF- and C-indexes in the exploratory set. A: Proportion of Metavir fibrosis (F) stages according to the maximum Youden index cut-off and the thresholds of 90% negative and positive predictive values for significant fibrosis with CSF-index. B: Proportion of Metavir F stages according to the maximum Youden index cut-off and the thresholds of 90% negative and positive predictive values for severe fibrosis with SF-index. C: Proportion of Metavir F stages according to the thresholds of 95% predictive values for cirrhosis with C-index.

FIG. 6: Proportion of Metavir fibrosis (F) stages as a function of CSF/SF classification (X axis with the rate of patients included in each class in italics), in the exploratory (A) and validation (B) sets. The bottom line indicates the fibrosis stage classification.

FIG. 7: Rates of correctly classified patients by fibrosis stage classifications as a function of Metavir fibrosis stages in the validation set. Hatched lines: single fibrosis tests, continuous lines: new fibrosis stage classifications derived from new combined fibrosis indexes. Because of the few number of F0 patients, F0 and F1 were pooled together.

FIG. 8: Rate of correctly classified patients by fibrosis stage classifications as a function of IQR/median ratio in the validation set. IQR is the interquartile range (values from 25 to 75% of patients).

### EXAMPLES

### Example 1: Construction of the Classification Based on the Percentiles

In a population of 1000 patients with chronic liver disease, a FibroMeter was carried out (resulting in a score result, ranging from 0 to 1) as well as a biopsy, resulting in a histological staging using the Metavir system, ranging from F0 to F4.

The population is discretized in 40 percentiles of 2.5% according to the score result.

The Table 6 is drawn, wherein the classes of histological reference are in columns and the previous percentile classes in lines.

TABLE 6

| | | Metavir F | | | | | |
|---|---|---|---|---|---|---|---|
| | | 0 | 1 | 2 | 3 | 4 | Number of patients |
| Percentiles | 1 | 8 | 25 | 3 | 0 | 0 | 36 |
| | 2 | 5 | 26 | 4 | 1 | 0 | 36 |
| | 3 | 2 | 25 | 6 | 3 | 0 | 36 |
| | 4 | 4 | 22 | 7 | 2 | 2 | 37 |
| | 5 | 3 | 22 | 9 | 2 | 0 | 36 |
| | 6 | 1 | 22 | 11 | 2 | 0 | 36 |
| | 7 | 2 | 23 | 9 | 3 | 0 | 37 |
| | 8 | 1 | 18 | 8 | 9 | 0 | 36 |
| | 9 | 2 | 11 | 12 | 9 | 2 | 36 |
| | 10 | 0 | 15 | 14 | 4 | 3 | 36 |
| | 11 | 0 | 11 | 14 | 5 | 7 | 37 |
| | 12 | 0 | 12 | 14 | 7 | 3 | 36 |
| | 13 | 0 | 7 | 12 | 12 | 5 | 36 |
| | 14 | 0 | 7 | 14 | 10 | 6 | 37 |
| | 15 | 1 | 8 | 13 | 10 | 4 | 36 |
| | 16 | 0 | 6 | 11 | 9 | 10 | 36 |
| | 17 | 0 | 4 | 8 | 16 | 9 | 37 |
| | 18 | 0 | 3 | 9 | 10 | 14 | 36 |
| | 19 | 0 | 5 | 3 | 9 | 19 | 36 |
| | 20 | 0 | 1 | 6 | 5 | 24 | 36 |
| | Total | 29 | 273 | 187 | 128 | 108 | 725 |

The most frequent histological stage in each percentile class is determined. In the following example, the most frequent stages per percentile are indicated in bold characters (Table 7).

TABLE 7

| | | Metavir F | | | | | |
|---|---|---|---|---|---|---|---|
| | | 0 | 1 | 2 | 3 | 4 | Number of patients |
| Percentiles | 1 | 8 | 25 | 3 | 0 | 0 | 36 |
| | 2 | 5 | 26 | 4 | 1 | 0 | 36 |
| | 3 | 2 | 25 | 6 | 3 | 0 | 36 |
| | 4 | 4 | 22 | 7 | 2 | 2 | 37 |
| | 5 | 3 | 22 | 9 | 2 | 0 | 36 |
| | 6 | 1 | 22 | 11 | 2 | 0 | 36 |
| | 7 | 2 | 23 | 9 | 3 | 0 | 37 |
| | 8 | 1 | 18 | 8 | 9 | 0 | 36 |
| | 9 | 2 | 11 | 12 | 9 | 2 | 36 |
| | 10 | 0 | 15 | 14 | 4 | 3 | 36 |
| | 11 | 0 | 11 | 14 | 5 | 7 | 37 |
| | 12 | 0 | 12 | 14 | 7 | 3 | 36 |
| | 13 | 0 | 7 | 12 | 12 | 5 | 36 |
| | 14 | 0 | 7 | 14 | 10 | 6 | 37 |
| | 15 | 1 | 8 | 13 | 10 | 4 | 36 |
| | 16 | 0 | 6 | 11 | 9 | 10 | 36 |
| | 17 | 0 | 4 | 8 | 16 | 9 | 37 |
| | 18 | 0 | 3 | 9 | 10 | 14 | 36 |
| | 19 | 0 | 5 | 3 | 9 | 19 | 36 |
| | 20 | 0 | 1 | 6 | 5 | 24 | 36 |
| | Total | 29 | 273 | 187 | 128 | 108 | 725 |

The rate of well classified patients in each line is then calculated.

On the first percentile line, the minimum number of contiguous columns (histological fibrosis stages) in order to reach a predefined minimum correct classification rate ≥80% is selected. Then, this process is expended to the next line until the correct classification rate is ≥80%.

When this correct classification rate declines, especially when it is lower than the predefined rate, one or contiguous column(s) with higher fibrosis stages (to the right hand of the table) are further selected to reach again the predefined minimum correct classification rate. Then, the correct classification rate for each histological stage on a bottom line is calculated (Table 8). It should be noted that the prevalence of F0 stage in usually low in this kind of study due to the low prevalence of liver biopsy in this stage. Therefore, the second preferred stage in the first class is F0 by convention to circumvent this bias.

Five fibrosis classes (this is an example): F0/1, F1/2, F1/2/3, F2/3/4, F3/4 (enclosed in heavyweight lines on Table 8) are obtained, with an overall accuracy of 85.8%.

The thresholds of each fibrosis class are obtained from the data base (Table 9).

TABLE 9

| | Score result |
|---|---|
| Equivalent | of fibrosis |
| Metavir F | (FibroMeter) |
| | 0 |
| F0/1 | |
| | 0.13925337 |
| F1 | |
| | 0.17132949 |
| F1/2 | |
| | 0.55542014 |
| F1/2/3 | |
| | 0.72255544 |
| F2/3 | |
| | 0.86852787 |
| F2/3/4 | |
| | 0.97262959 |
| F3/4 | |
| | 1 |

### Example 2: Example of Classification Based on the Percentiles (FibroMeter^{3G})

### Methods

### Study Design

We recruited different populations with liver biopsy to evaluate the different diagnostic means. Thus, populations #1, #2 and #3 included blood tests. The three populations were separately analysed due to their initial different designs and to evaluate the accuracy robustness given these differences.

### Populations

Patients with chronic HCV hepatitis, liver biopsy, blood tests and available Fibroscan were consecutively recruited in different populations #1 to #3 described in Table 10.

TABLE 10
Main characteristics of HCV populations.

| | | | Liver | | | | | | | |
|---|---|---|---|---|---|---|---|---|---|---|
| | | | biopsy | | | | | | | |
| | Study | Patients | length | Blood | | Metavir F prevalence (%) | | | | |
| Population # | name | (n) | (mm) | tests | FS | 0 | 1 | 2 | 3 | 4 |
| 1 | Sniff 17 | 1056 | 21 ± 8 | x | — | 4.4 | 43.5 | 27.0 | 14.0 | 11.2 |
| 2 | Fibrostar | 458 | 25 ± 8 | x | x | 6.7 | 45.1 | 17.9 | 15.6 | 14.8 |
| 3 | Vindiag 7 | 349 | 25 ± 9 | x | x | 1.4 | 30.7 | 35.5 | 20.6 | 11.7 |

x: test performed,
FS: Fibroscan

Each population had different characteristics and fibrosis assessments. Inclusion and exclusion criteria are detailed in previous publications or below for new populations. Briefly, patients did not receive antiviral or known anti-fibrotic treatments. Liver biopsy, blood withdrawal and Fibroscan, when available, were performed within a maximum of 6-month time interval.

Population #1 included 1056 patients provided by five centers participating in the Sniff 17 study (Cales P et al., Liver Int 2008; 28:1352-62). Thus, individual patient data were available from five centers, independent for study design, patient recruitment, blood marker determination and interpretation of liver histology by an expert pathologist. Blood and pathological determinations were not centralized.

Population #2 included 458 patients provided by 19 centers participating in the Fibrostar study (Zarski J P et al., Hepatology 2009; 50:1061A). Blood determination and liver interpretation were centralized. Liver specimens were read by two senior experts, one of whom was from the Metavir group.

Population #3 included 349 patients provided by three centers participating in the Vindiag 7 study (Boursier J et al., J Hepatol 2010; 52:S405). Blood and pathological (one senior expert in each center) determinations were not centralized.

### Diagnostic Means

Fibrosis was staged in liver biopsy according to Metavir staging (The French METAVIR Cooperative Study Group, Hepatology 1994; 20:15-20) in all patients. This fibrosis stage classification was used as the reference for the calculation of accuracy. Blood tests were determined in all studies. We only evaluated here FibroMeter™ (Cales P et al., Hepatology 2005; 42:1373-81, Biolivescale, Angers, France).

### Fibrosis Classifications

We distinguished as fibrosis degrees the histological fibrosis stages and the fibrosis classes provided by non-invasive tests and including one or several fibrosis stages. Several fibrosis classifications were evaluated:
The histological fibrosis stage classification into 5 F_{M} stages, as determined on a liver specimen by a pathologist. This was the reference for accuracy.
The binary diagnosis of significant fibrosis (2 classes) determined either on liver specimen or by the diagnostic cut-off in non-invasive tests. This is the usual diagnostic target of non-invasive tests and thus served as comparator for the detailed classifications. Indeed, as it was expected that a more detailed classification would result in decreased accuracy, this binary accuracy allowed to evaluate this putative accuracy loss.
The fibrosis class classification corresponding to the classification based on percentiles described in the present invention.

### Results

FibroMeter^{3G} shows a significant increase in correct classification rate of fibrosis class classification compared to significant fibrosis diagnosis.

### Population #1

### Classification Accuracy

The accuracy of fibrosis class classification by FibroMeter^{3G} was 86.9% vs. 77.9% for binary diagnosis of significant fibrosis (11.6% relative increase) (Table 11).

TABLE 11
Rates of correct classification by blood tests (%, italicized entries)
as a function of fibrosis classification in population #1.

| | Significant fibrosis | | |
|---|---|---|---|
| | (F ≥ 2) | Fibrosis classes | p^{a} |
| FibroMeter^{3G} (FM^{3G}) | 77.9 | 86.9 | <10⁻³ |

^{a}By McNemar test (pair)

### Populations #2 and 3

In population #2 (and #3), the accuracy of the fibrosis class classifications was 77.1% (83.4%) for FibroMeter^{3G} (Table 12).

TABLE 12
Rates of correct classification by non-invasive means
(%, italicized entries) as a function of fibrosis
classification in populations #2 and #3.

| | Population #2 | | | Population #3 | | |
|---|---|---|---|---|---|---|
| | | Fibrosis | | | Fibrosis | |
| | Significant | class | | Significant | class | |
| | fibrosis | classi- | | fibrosis | classi- | |
| | (F_{M} ≥ 2) | fication | p^{a} | (F_{M} ≥ 2) | fication | p^{a} |
| FibroMeter^{3G} | 74.0 | 77.1 | 0.255 | 76.8 | 83.4 | 0.011 |
| (FM^{3G}) | | | | | | |

^{a}By McNemar test (pair)

### Example 3: Example of Classification Based on Percentiles (FibroMeter+Fibroscan)

### Methods

### Study Design

We recruited different populations with liver biopsy to evaluate the different diagnostic means. Thus, populations #1, #2 and #3 included blood tests. The three populations were separately analysed due to their initial different designs and to evaluate the accuracy robustness given these differences.

The study aims at evaluating method providing binary diagnosis, such as SAFE and BA, with cross-checked FibroTest with APRI or Fibroscan, with comparison to the new, non-invasive FibroMeter+Fibroscan classification (based on percentiles).

### Populations

Patients with chronic HCV hepatitis, liver biopsy, blood tests and available Fibroscan were consecutively recruited in different populations #1 to #3 described in Table 13.

TABLE 13
Main characteristics of populations.

| | | | Liver | | | | | | | |
|---|---|---|---|---|---|---|---|---|---|---|
| | | | biopsy | | | | | | | |
| | Study | Patients | length | Blood | | Metavir F prevalence (%) | | | | |
| Population # | name | (n) | (mm) | tests | FS | 0 | 1 | 2 | 3 | 4 |
| 1 | Sniff 32 | 1056 | 21 ± 8 | x | — | 4.4 | 43.5 | 27.0 | 14.0 | 11.2 |
| 2 | Fibrostar + | 458 | 25 ± 9 | x | x | 4.0 | 37.7 | 25.8 | 17.6 | 15 |
| | Vindiag7 | | | | | | | | | |

x: test performed,
FS: Fibroscan

Each population had different characteristics and fibrosis assessments. Inclusion and exclusion criteria are detailed in previous publications or below for new populations. Briefly, patients did not receive antiviral or known anti-fibrotic treatments. Liver biopsy, blood withdrawal and Fibroscan, when available, were performed within a maximum of 6-month time interval.

Population #1 included 1056 patients provided by five centers participating in the Sniff 32 study (Cales P et al., Liver Int 2008; 28:1352-62). Thus, individual patient data were available from five centers, independent for study design, patient recruitment, blood marker determination and interpretation of liver histology by an expert pathologist. Blood and pathological determinations were not centralized.

Population #2 included 458 patients provided by 19 centers participating in the Vindiag 7 (Boursier et al., Am. J. Gastroenterol 2011; 106; 1255-1263) and in Fibrostar study (Zarski J P et al., J Hepatol 2012; 56:55-62). Blood determination and liver interpretation were centralized. Liver specimens were read by two senior experts, one of whom was from the Metavir group.

### Diagnostic Means

Fibrosis was staged in liver biopsy according to Metavir staging (The French METAVIR Cooperative Study Group, Hepatology 1994; 20:15-20) in all patients. This fibrosis stage classification was used as the reference for the calculation of accuracy. Blood tests were determined in all studies. We only evaluated here FibroMeter™ (Cales P et al., Hepatology 2005; 42:1373-81, Biolivescale, Angers, France).

Liver Stiffness Evaluation. FibroScan was available in the VINDIAG 7 and FIBROSTAR studies. FibroScan examinations were performed under fasting conditions by an experienced observer (>50 examinations before the study), blinded for patient data. Examination conditions were those recommended by the manufacturer. 19 FibroScan examinations were stopped when 10 valid measurements were recorded.

Results (in kilopascals) were expressed as the median of all valid measurements. A FibroScan result was considered reliable when the interquartile range (IQR)/median ratio (IQR/M) was <0.21.

### Fibrosis Classifications

We distinguished as fibrosis degrees the histological fibrosis stages and the fibrosis classes provided by non-invasive tests and including one or several fibrosis stages. Several fibrosis classifications were evaluated:
The histological fibrosis stage classification into 5 F_{M} stages (Metavir system), as determined on a liver specimen by a pathologist. This was the reference for accuracy.
The binary diagnosis of significant fibrosis (2 classes) determined either on liver specimen or by the diagnostic cut-off in non-invasive tests. This is the usual diagnostic target of non-invasive tests and thus served as comparator for the detailed classifications. Indeed, as it was expected that a more detailed classification would result in decreased accuracy, this binary accuracy allowed to evaluate this putative accuracy loss.
The fibrosis class classification corresponding to the classification based on percentiles described in the present invention.

### Results

TABLE 14
Comparison of Diagnostic Accuracies (%) and Rates of Required Liver Biopsy
(LB, %) Between Decision-Making Algorithms Constructed for a Binary Diagnosis of
Liver Fibrosis (Bold Values) and Either Successive Algorithms or the New FM + FS
Classification, as a Function of Study Population

| | | Population | | | | | |
|---|---|---|---|---|---|---|---|
| Fibrosis algorithm | | All | | #1 | | #2 | |
| type | Name | Accuracy | LB | Accuracy | LB | Accuracy | LB |
| Decision making | SAFE for F ≥ 2 | 94.6 | 64.0 | 96.0 | 68.8 | 92.5 | 57.0 |
| algorithm | SAFE for F4 | 89.5 | 6.4 | 90.7 | 6.2 | 87.6 | 6.7 |
| | SAFE for F ≥ 2 and | 97.0 | 85.2 | 97.8 | 87.6 | 95.8 | 81.7 |
| | F4 | | | | | | |
| | BA for F ≥ 2 | | | | | 88.3 | 34.6 |
| | BA for F4 | | | | | 94.2 | 24.6 |
| Successive | Successive SAFE | 87.3* | 70.8* | 89.6† | 75.7* | 84.1* | 63.8* |
| algorithm | Successive BA | | | | | 84.7‡ | 49.8‡ |
| Non-invasive | FM + FS | | | | | 86.7§ | 0.0 |
| classification | classification | | | | | | |
| fibrosis | | | | | | | |

*P ≤ 10−3 versus SAFE for F ≥ 2 or SAFE for F4
†P ≤ 10−3 versus SAFE for F ≥ 2 and P = 0.059 versus SAFE for F4
‡P ≤ 10−3 versus BA for F ≥ 2 or BA for F4
§P > 0.118 versus Successive SAFE or Successive BA

The most accurate synchronous combination of FibroScan with a blood test (FibroMeter) provided a new detailed (six classes) classification (FM+FS). Successive SAFE had a significantly (P<10⁻³) lower diagnostic accuracy (87.3%) than individual SAFE for F≥2 (94.6%) or SAFE for F4 (89.5%), and required significantly more biopsies (70.8% versus 64.0% or 6.4%, respectively, P<10⁻³). Similarly, successive BA had significantly (P<10⁻³) lower diagnostic accuracy (84.7%) than individual BA for F≥2 (88.3%) or BA for F4 (94.2%), and required significantly more biopsies (49.8% versus 34.6% or 24.6%, respectively, P<10⁻³). The diagnostic accuracy of the FM+FS classification (86.7%) was not significantly different from those of successive SAFE or BA. However, this new classification required no biopsy.

Conclusion: SAFE and BA for significant fibrosis or cirrhosis are very accurate. However, in clinical practice, the significant fibrosis algorithm and the cirrhosis algorithm have to be used successively, which induces a significant decrease in diagnostic accuracy and a significant increase in the rate of required liver biopsy. A new fibrosis classification that synchronously combines two fibrosis tests was as accurate as successive SAFE or BA, while providing an entirely noninvasive (0% liver biopsy) and more precise (six versus two or three fibrosis classes) fibrosis diagnosis.

### Example 4: Example of Classification Based on Percentiles (Cirrhosis)

Cirrhosis diagnosis is a clinically important diagnostic target. The method of the invention improves the accuracy (% of well-classified patients) and precision (Metavir fibrosis stage number per test class) of non-invasive fibrosis diagnosis focused on cirrhosis.

### Methods:

Populations—All patients had chronic hepatitis C, liver biopsy and 6 blood tests.

TABLE 15
Main characteristics of HCV populations.

| | | | Liver | | | | | | | |
|---|---|---|---|---|---|---|---|---|---|---|
| | | | biopsy | | | | | | | |
| | Study | Patients | length | Blood | | Metavir F prevalence (%) | | | | |
| Population # | name | (n) | (mm) | tests | FS | 0 | 1 | 2 | 3 | 4 |
| 1 | Sniff 17 | 1056 | 21 ± 8 | x | — | 4.4 | 43.5 | 27.0 | 14.0 | 11.2 |
| 2 | | 729 | | x | x | 4.0 | 37.7 | 25.8 | 17.6 | 15.0 |

x: test performed,
FS: Fibroscan

Test Combination Development—

We compared different combinations of blood tests and Fibroscan, combined by single logistic regression. This method showed that CirrhoMeter^{2G} or FibroMeter^{2G} and Fibroscan were independent predictors of cirrhosis.

Fibrosis Classification—

For non-invasive tests, we used the fibrosis classification based on percentiles. We thus developed a new fibrosis classification for Fibroscan and/or CirrhoMeter^{2G} or FibroMeter^{2G} by determining specific test thresholds.

### Single Fibrosis Tests

### Binary Cirrhosis Diagnosis

The AUROC of CirrhoMeter^{2G} was 0.919 (95% CI: 0.893-0.945) in the derivation population #1 and 0.857 (0.813-0.900), p<0.001, in the validation population #2. Also in this latter population, the AUROC of Fibroscan was 0.905 (0.871-0.938), p=0.041. CirrhoMeter and Fibroscan had respectively: binary cirrhosis diagnosis, accuracy: 89.4% vs. 89.7% (p=0.902)

Sensitivity and specificity respectively for CirrhoMeter^{2G} and Fibroscan were as follows: 36.5% vs. 58.3% (p<0.001) and 98.1% vs. 94.9% (p=0.003).

### Fibrosis Classification

We developed fibrosis classifications for CirrhoMeter and/or Fibroscan including 6 classes; their performance was globally evaluated with a precision index weighted on accuracy (IPA) then on biopsy (IPAB).

### Comparison of CirrhoMeter^{2G} and Fibroscan

Using similar a posteriori thresholds, the accuracies were, CirrhoMeter^{2G}: 88.2% vs. Fibroscan: 88.8% (p=0.773). Finally, the diagnostic characteristics of these classifications were globally not significantly different except for the precision/accuracy ratio (IPA), which was significantly lower, i.e., better, with Fibroscan (2.31 vs. 2.47).

### Fibrosis Test Combination

### Combination Description

FibroMeter^{2G}+Fibroscan constructed for significant fibrosis (called hereafter “FibroMeter^{2G}+Fibroscan for FM≥2”) provided the following characteristics: AUROC: 0.922 (0.893-0.950), accuracy: 91.3%, sensitivity: 57.3% and specificity: 96.9%.

We developed a fibrosis classification for FibroMeter^{2G}+Fibroscan for FM≥2.

### Comparison Between Combination and Single Fibrosis Tests

Binary Cirrhosis Diagnosis—

The difference in AUROCs between Fibroscan (0.905) and FibroMeter^{2G}+Fibroscan for FM≥2 (0.922) was not significant (p=0.078).

Fibrosis Classification—

FibroMeter^{2G}+Fibroscan for FM≥2 had a significantly better precision/accuracy index than single tests (p<0.001).

Sensitivity for cirrhosis in the F4 class was: CirrhoMeter^{2G}: 14.6%, Fibroscan: 27.1%, and FibroMeter^{2G}+Fibroscan for FM≥2: 29.5%, which is an apparent decrease compared to the sensitivity previously shown by the binary diagnoses of CirrhoMeter2G (44.8%) or Fibroscan (53.1%). However, the overall sensitivity of the classifications for cirrhosis was 82.3%, 83.3%, and 93.7%, respectively.

Finally, the positive predictive value for cirrhosis of the F4 class was 82.4%, 78.8%, and 84.8%, respectively.

The cirrhosis affirmation/exclusion prediction by FibroMeter+Fibroscan was twice (34.6%) that of the best single test (16.2%, p<0.001).

### Algorithms Including Liver Biopsy

### Development

The limit of the previous fibrosis classifications is that they provide intermediate classes in cirrhosis diagnosis (F3±1 and F3/4 classes). This grey-zone limit may be circumvented by performing liver biopsy when necessary. High performance (≥92%) can thus be achieved not only for overall accuracy, but also—and more importantly—for cirrhosis sensitivity, by performing liver biopsy in ≤30% of patients.

Comparison with Other Algorithms

The main advantages of the FibroMeter^{2G}+Fibroscan for FM≥2 algorithm compared to successive SAFE or BA were slightly higher cirrhosis sensitivity, a marked reduction in liver biopsy rate and a substantially increased precision. Therefore, the precision/accuracy/biopsy ratio (IPAB) was significantly different between all tests (p<0.001 by paired Friedman test) with the following decreasing rank order: FibroMeter2G+Fibroscan for FM≥2≈Fibroscan<CirrhoMeter2G<successive BA<successive SAFE<SAFE for cirrhosis<BA for cirrhosis.

The FibroMeter+Fibroscan combination improves overall precision, and sensitivity and prediction for cirrhosis. This strategy permits a precise diagnosis of cirrhosis and other fibrosis stages either fully non-invasively or with low (<30%) biopsy rate.

### Example 5: Classification Based on the Combination of RDIs

### Methods

### Patients

Exploratory Set—

Patients with CHC hospitalized for a percutaneous liver biopsy were prospectively enrolled from March 2004 to September 2008 in 3 tertiary centers in France (Angers, Bordeaux, and Grenoble). Patients with cirrhosis complications (ascites, variceal bleeding, systemic infection, hepatocellular carcinoma) were not included. Blood fibrosis tests and Fibroscan were performed in the week preceding biopsy. All patients gave their informed consent. The study protocol conformed to the ethical guidelines of the current Declaration of Helsinki and received approval from the local Ethics committee.

Validation Set—

The validation set corresponded to the multicenter population of the FIBROSTAR study promoted by the French National Agency for research in AIDS and hepatitis (Zarski J P. et al., J Hepatol 2010; 52:S175). This study prospectively included 512 patients with CHC. All patients had liver biopsy, blood fibrosis tests and Fibroscan. Patients included in both the exploratory set and the FIBROSTAR study were excluded from the validation set.

### Methods

Histological Assessment—

Liver fibrosis was evaluated according to Metavir staging. Significant fibrosis was defined as Metavir stages F≥2, severe fibrosis as Metavir F≥3, and cirrhosis as F4. In the exploratory set, liver fibrosis was evaluated by two senior experts with a consensus reading at Angers, and by a senior expert at Bordeaux and Grenoble. In the FIBROSTAR study, liver fibrosis was centrally evaluated by two senior experts with a consensus reading in cases of discordance. Fibrosis staging was considered as reliable when liver specimen length was ≥15 mm and/or portal tract number ≥8 (Nousbaum J B. et al., Gastroenterol Clin Biol 2002; 26:848-78). Liver biopsy was used as the reference for the liver fibrosis evaluations by non-invasive tests.

Fibrosis Blood Tests—

The following blood tests were calculated according to published or patented formulas: Fibrotest (Castera L. et al., Gastroenterology 2005; 128:343-50), FibroMeter (Leroy V. et al., Clin Biochem 2008; 41:1368-76), Hepascore (Adams L A. et al., Clin Chem 2005; 51:1867-73), FIB-4 (Sterling R K. et al., Hepatology 2006; 43:1317-25), and APRI (Wai C T. et al., Hepatology 2003; 38:518-26). All blood assays were performed in the same laboratories of each center, or centralized in the FIBROSTAR study.

Liver Stiffness Evaluation—

Fibroscan (EchoSens, Paris, France) examination was performed by an experienced observer (>50 examinations before the study), blinded for patient data. Examination conditions were those recommended by the manufacturer (Castera L. et al., J Hepatol 2008; 48:835-47). Fibroscan examination was stopped when 10 valid measurements were recorded. Results (kilopascals) were expressed as the median and the interquartile range of all valid measurements. Fibroscan results were considered as reliable when the ratio interquartile range/result (IQR/median) was <0.21 (Lucidarme D. et al., Hepatology 2009; 49:1083-9).

### Statistical Analysis

Quantitative variables were expressed as mean±standard deviation. The diagnostic cut-offs of fibrosis tests were calculated according to the highest Youden index (sensitivity+specificity−1), unless otherwise specified.

### Fibrosis Stage Classifications

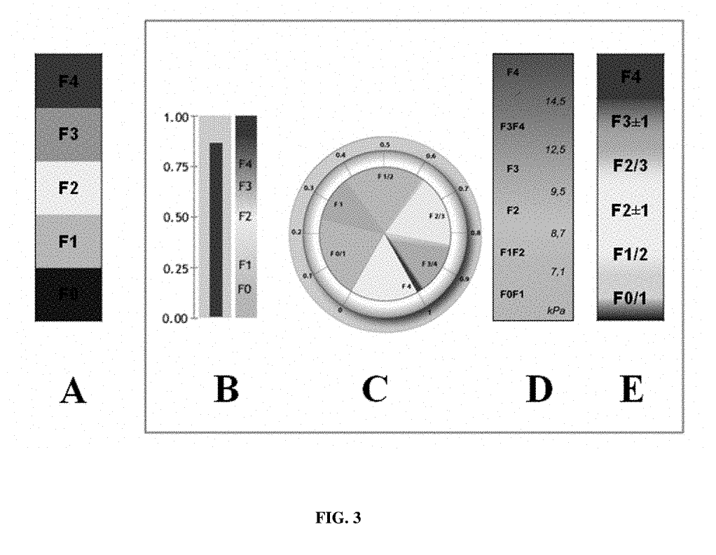

We evaluated the accuracy of Fibrotest, FibroMeter, and Fibroscan fibrosis stage classifications (FIG. 3). Fibrotest, Fibroscan, and FibroMeter classifications were those previously published (Leroy V. et al., Clin Biochem 2008; 41:1368-76, de Ledingen V. et al., Gastroenterol Clin Biol 2008; 32:58-67, Poynard T. et al., Comp Hepatol 2004; 3:8). Fibrotest classification includes 8 classes (F0, F0/1, F1, F1/2, F2, F3, F3/4, F4), Fibroscan classification: 6 classes (F0/1, F1/2, F2, F3, F3/4, F4), and FibroMeter classification: 6 classes (F0/1, F1, F1/2, F2/3, F3/4, F4).

### New Fibrosis Stage Classification

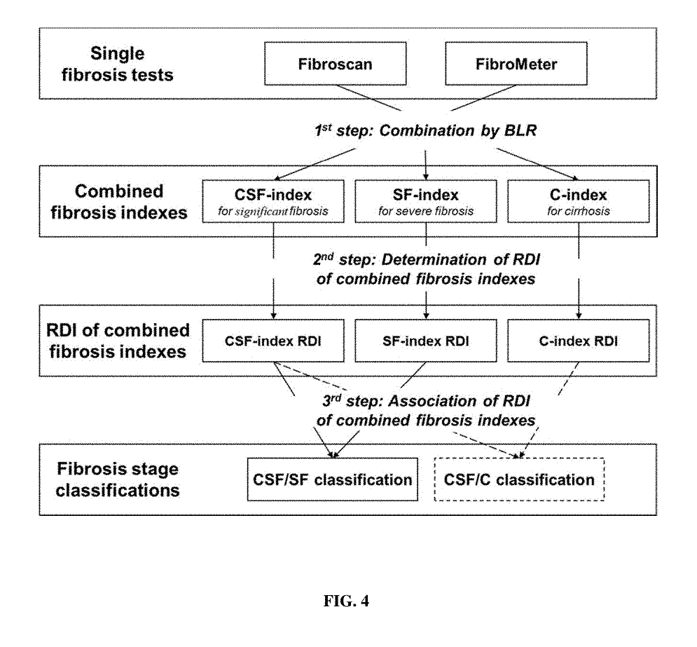

The 3-step procedure used to implement the new fibrosis stage classification is detailed in the FIG. 4.

1^{st} Step: New Combined Fibrosis Indexes—

To identify the best combination of single fibrosis tests for the diagnosis of significant fibrosis, we performed a stepwise binary logistic regression repeated on 1,000 bootstrap samples in the exploratory set. Independent variables tested were the 5 blood fibrosis tests and Fibroscan. The bootstrap method consists of a repeated sampling (with replacement) from the original entire dataset, followed by a stepwise logistic regression procedure in each subsample (1,000 subsamples here). The most frequently (>50%) selected single fibrosis tests among the 1,000 analyses were then included in a single binary logistic regression performed in the whole population of the exploratory set. Using the regression score of this multivariate analysis, we constructed a new combined fibrosis index for clinically significant fibrosis called “CSF-index”, ranging from 0 to 1. We also constructed combined fibrosis indexes for the diagnosis of severe fibrosis (SF-index) and cirrhosis (C-index) using the same process.

2^{nd} Step: Reliable Diagnosis Intervals—

RDIs correspond to the intervals of fibrosis test values where the individual diagnostic accuracy is considered sufficiently reliable for clinical practice. This method has been previously described (Cales P. et al., Liver Int 2008; 28:1352-62). Briefly, we first calculated the 90% negative and positive predictive value thresholds for significant fibrosis of the CSF-index. These 2 thresholds determined 3 intervals of CSF-index values: a low interval (from 0 to the 90% negative predictive value threshold) where the non-invasive diagnosis was consequently “F0/1”; a high interval (from the 90% positive predictive value threshold to 1) where the diagnosis was “F≥2”; and an intermediate interval between the two thresholds. The intermediate interval was then divided into two new intervals according to the diagnostic cut-off corresponding to the highest Youden index. In each of these two new intermediate intervals, the non-invasive diagnosis corresponded to the combined Metavir F stages having ≥90% prevalence (for example: F1/2 for the interval between the 90% negative predictive value threshold and the highest Youden index cut-off). Finally, the 4 RDI that were obtained provided ≥90% diagnostic accuracy by definition.

We also calculated the RDIs of SF-index and C-index in the same way. Because SF-index was developed for the diagnosis of severe fibrosis, its 90% negative and positive predictive value thresholds and its highest Youden index cut-off were determined for this diagnostic target. For C-index, we calculated the thresholds for cirrhosis according to the 95% predictive values due to the clinical importance of cirrhosis diagnosis.

3^{rd} Step: New Fibrosis Stage Classifications—

A new fibrosis stage classification was derived by associating RDIs for CSF- and SF-indexes. For example, if CSF-index provided a reliable diagnosis of “F≥2” and SF-index a reliable diagnosis of “F2±1”, the ensuing diagnosis of the new fibrosis stage classification was “F2/3”. Another fibrosis stage classification was derived by associating RDIs for CSF- and C-indexes.

Statistical softwares were SPSS, version 17.0 (SPSS Inc., Chicago, Ill., USA) and SAS 9.1 (SAS Institute Inc., Cary, N.C., USA).

### Results

### Patients

The exploratory and validation sets included 349 and 380 patients respectively. The characteristics of both sets are detailed in Table 16.

TABLE 16
Patient characteristics at inclusion.

| | | Set | | |
|---|---|---|---|---|
| | All patients | Exploratory | Validation | p |
| Patients (n) | 729 | 349 | 380 | — |
| Males (%) | 61.3 | 60.2 | 62.4 | 0.531 |
| Age (years) | 51.7 ± 11.2 | 52.1 ± 11.2 | 51.3 ± 11.2 | 0.347 |
| Metavir (%): | | | | <0.001 |
| F0 | 4.0 | 1.4 | 6.3 | |
| F1 | 37.7 | 30.7 | 44.2 | |
| F2 | 25.8 | 35.5 | 16.8 | |
| F3 | 17.6 | 20.6 | 14.7 | |
| F4 | 15.0 | 11.7 | 17.9 | 0.020 |
| Significant fibrosis | 58.3 | 67.9 | 49.5 | <0.001 |
| (%) | | | | |
| Reliable biopsy (%) | 93.5 | 92.6 | 94.2 | 0.391 |
| Fibroscan result | 10.0 ± 7.9 | 9.9 ± 8.1 | 10.1 ± 7.7 | 0.755 |
| (kPa) | | | | |
| IQR/median <0.21 | 66.9 | 66.2 | 67.6 | 0.700 |
| (%) | | | | |

kPa: kilopascal;
IQR: interquartile range

Among the two sets, 93.5% of liver biopsies were considered as reliable.

### Development of New Fibrosis Stage Classifications

### 1^{st} Step: New Combined Fibrosis Indexes

For each diagnostic target of liver fibrosis, Fibroscan and FibroMeter were single fibrosis tests the most frequently selected by the stepwise binary logistic regression repeated on the 1000 bootstrap samples. These 2 fibrosis tests were independent variables in logistic models ran in the exploratory set and thus provided 3 new combined fibrosis indexes for 3 diagnostic targets: CSF-index for significant fibrosis, SF-index for severe fibrosis, and C-index for cirrhosis. CSF-index had a significantly higher AUROC than its composite tests, i.e., FibroMeter or Fibroscan, in the exploratory set (Table 17).

SF-index and C-index also had higher AUROCs than FibroMeter or Fibroscan in the exploratory set, but the difference was significant only with FibroMeter.

2^{nd} Step: Reliable Diagnosis Intervals

CSF-Index (Diagnostic Target: Significant Fibrosis)—

CSF-index was divided into 4 reliable diagnosis intervals. The extreme intervals were the traditional intervals of ≥90% negative (NPV) or positive (PPV) predictive values for significant fibrosis. CSF-index included 9.2% of patients in the ≥90% NPV interval (CSF-index value ≥0 and ≤0.248) and 46.1% in the ≥90% PPV interval (CSF-index value ≥0.784 and ≤1). Thus, CSF-index displayed a reliable diagnosis of significant fibrosis with ≥90% accuracy in 55.3% of patients versus 33.8% with Fibroscan (p<0.001) and 55.6% with FibroMeter (p=1.00, Table 18).

TABLE 18
Rate of patients included in the intervals of reliable diagnosis defined by the
≥90% negative (NPV) and positive (PPV) predictive values for significant fibrosis
(Metavir F ≥ 2) or severe fibrosis (Metavir F ≥ 3), and the ≥95% predictive values for
cirrhosis (Metavir F4), as a function of diagnostic target and fibrosis test, and according to
patient group.

| | | Metavir F ≥ 2 | | Metavir F ≥ 3 | | Metavir F4 | |
|---|---|---|---|---|---|---|---|
| | Fibrosis | | Correctly | | Correctly | | Correctly |
| Set | test | Patients^{a} | classified^{b} | Patients^{a} | classified^{b} | Patients^{a} | classified^{b} |
| Exploratory | FibroMeter | 55.6 | 89.7 | 41.8 | 89.7 | 65.9 | 94.8 |
| | Fibroscan | 33.8 | 90.7 | 46.4 | 90.1 | 87.4 | 94.8 |
| | Combined | 55.3 | 90.2 | 49.9 | 89.7 | 89.7 | 94.9 |
| | index^{c} | | | | | | |
| Validation | FibroMeter | 48.8 | 72.7 | 47.0 | 94.2 | 64.2 | 97.2 |
| | Fibroscan | 38.2 | 77.0 | 46.7 | 93.5 | 85.2 | 93.2 |
| | Combined | 49.1 | 85.2 | 58.5 | 95.9 | 87.3 | 93.8 |
| | index^{c} | | | | | | |
| All | FibroMeter | 52.3 | 82.0 | 44.3 | 92.0 | 65.1 | 95.9 |
| | Fibroscan | 35.9 | 83.6 | 46.5 | 91.8 | 86.3 | 94.0 |
| | Combined | 52.3 | 87.9 | 54.1 | 92.9 | 88.5 | 94.3 |
| | index^{c} | | | | | | |

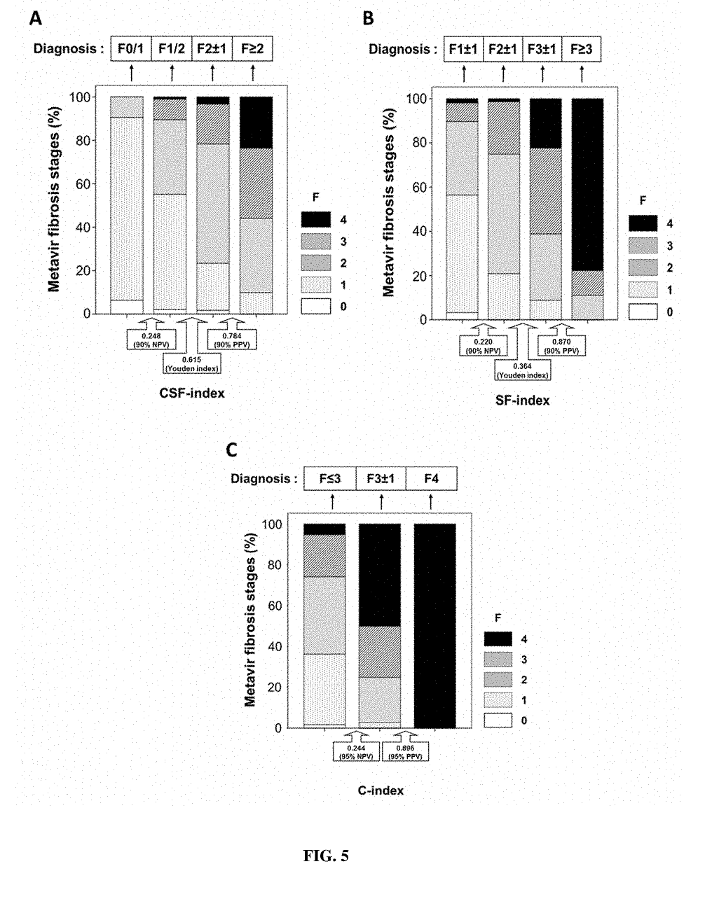

The indeterminate interval (between CSF-index values >0.248 and <0.784) was then divided into 2 new intervals according to the diagnostic cut-off corresponding to the maximum Youden index (0.615). 87.5% of the patients included in the lower new interval (>0.248-<0.615) had F1/2 stages according to liver biopsy results, and 95.0% of patients included in the higher new interval (≥0.615 and <0.784) had F1/2/3 stages (FIG. 5A). Finally, CSF-index provided 4 RDIs whose F classification was: F0/1, F1/2, F2±1, and F≥2. The diagnostic accuracy of these RDIs was 90.3% (FIG. 5A).

FibroMeter provided the same 4 RDIs with 89.4% diagnostic accuracy (p=0.664 vs CSF-index).

SF-Index (Diagnostic Target: Severe Fibrosis)—

SF-index was also divided into 4 RDIs. The extreme intervals were the traditional intervals of ≥90% negative or positive predictive values for severe fibrosis. SF-index included 44.7% of patients in the ≥90% NPV interval (SF-index value ≥0 and ≤0.220) and 5.2% in the ≥90% PPV interval (SF-index value ≥0.870 and ≤1). Thus, SF-index displayed a reliable diagnosis of significant fibrosis with ≥90% accuracy in 49.9% of patients (Table 18) versus 41.8% with FibroMeter (p<0.001) and 46.4% with Fibroscan (p=0.235). By dividing the indeterminate interval of SF-index according to the diagnostic cut-off (maximum Youden index: 0.364), SF-index provided 4 RDI (F1±1, F2±1, F3±1, F≥3; FIG. 5B) with 92.0% diagnostic accuracy.

Fibroscan provided the same 4 RDIs with 91.1% diagnostic accuracy (p=0.728 vs SF-index).

C-Index (Diagnostic Target: Cirrhosis)—

C-index included 87.7% of patients in the ≥95% NPV interval for cirrhosis (C-index value ≥0 and ≤0.244), and 2.0% in the ≥95% PPV interval for cirrhosis (C-index value ≥0.896 and ≤1). Thus, C-index displayed a reliable diagnosis of cirrhosis with ≥95% accuracy in 89.7% of patients (Table 18) versus 65.9% with FibroMeter (p<0.001) and 87.4% with Fibroscan (p=0.096). Dividing the indeterminate interval according to the diagnostic cut-off did not distinguish two different groups. Finally, C-index provided 3 RDIs (F≤3, F3±1, F4) with 95.1% diagnostic accuracy (FIG. 5C).

In conclusion, by using the thresholds of 90% predictive values for significant fibrosis and the diagnostic cut-off corresponding to the maximum Youden index, CSF-index provided 4 RDIs (F0/1, F1/2, F2±1, F≥2), which provided 90.3% diagnostic accuracy. By using the same method for severe fibrosis, SF-index provided 4 RDIs (F1±1, F2±1, F3±1, F≥3) with 92.0% diagnostic accuracy. Finally, by using the thresholds of 95% predictive values for cirrhosis, C-index provided 3 RDIs (F<3, F3±1, F4) with 95.1% diagnostic accuracy.

### 3^{rd} Step: New Fibrosis Stage Classifications

The first classification (CSF/SF classification) was derived from the association of CSF- and SF-index RDIs (Table 19).

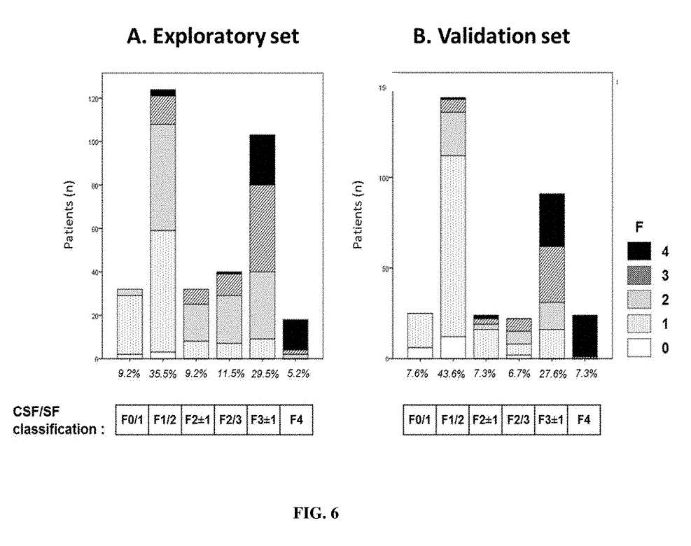

CSF/SF classification included 6 classes (F0/1, F1/2, F2±1, F2/3, F3±1, F4) and provided 87.7% diagnostic accuracy in the exploratory set (FIG. 6A).

The second classification (CSF/C classification) was derived from CSF- and C-index RDIs (Table 19). CSF/C classification also included 6 classes (F0/1, F1/2, F2±1, F2/3, F3±1, F4) and provided 86.5% diagnostic accuracy (p=0.503 vs CSF/SF classification, Table 20).

TABLE 20
Diagnostic accuracy (% of correctly classified patients) of fibrosis stage
classifications as a function of patient group.

Set

| Classification | All | Exploratory | Validation | p^{a} |
|---|---|---|---|---|
| CSF/SF | 86.7 | 87.7 | 85.8 | 0.461 |
| CSF/C | 84.4 | 86.5 | 82.1 | 0.113 |
| FibroMeter | 68.7 | 67.6 | 69.7 | 0.550 |
| Fibroscan | 58.7 | 54.4 | 63.3 | 0.020 |
| Fibrotest | 38.8 | 33.5 | 43.9 | 0.005 |

### Association of Combined Fibrosis Indexes RDIs or Single Fibrosis Tests RDIs?

As previously shown, the accuracies of RDIs from combined fibrosis indexes and their composite single fibrosis tests were not significantly different (i.e., the FibroMeter RDIs for significant fibrosis vs that of CSF-index, and the Fibroscan RDIs for severe fibrosis vs that of SF-index). Therefore, we implemented a third classification (FM/FS classification) that was derived from the FibroMeter RDIs for significant fibrosis and the Fibroscan RDIs for severe fibrosis. Results of FibroMeter and Fibroscan RDIs were discordant in 2 patients, who thus had indeterminate diagnoses. FM/FS classification ultimately included 7 classes (F0/1, F1, F1/2, F2, F2/3, F3±1, F4) and provided 82.8% diagnostic accuracy (p=0.006 vs CSF/SF classification). However, diagnostic accuracy of FM/FS classification dramatically decreased to 69.4% in the validation set (p<0.001 vs CSF/SF and CSF/C classifications).

### Validation of the New Fibrosis Stage Classifications

The diagnostic accuracies of CSF-index, SF-index, and C-index RDIs were not significantly different between the exploratory and the validation sets, with respectively: 90.3% vs 86.7% (p=0.142), 92.0% vs 91.5% (p=0.827), and 95.1% vs 94.5% (p=0.731). Similarly, diagnostic accuracies of CSF/SF and CSF/C classifications were not significantly different between the 2 sets (Table 20).

In the validation set, CSF/SF classification provided a significantly higher diagnostic accuracy (85.8%) than CSF/C classification and those of single fibrosis tests (p<0.008, Table 20). FIG. 6B shows the proportion of Metavir fibrosis stages as a function of CSF/SF classification. According to diagnostic accuracy in the validation set, classification ranking was: CSF/SF>CSF/C>FibroMeter>Fibroscan>Fibrotest (Table 20).

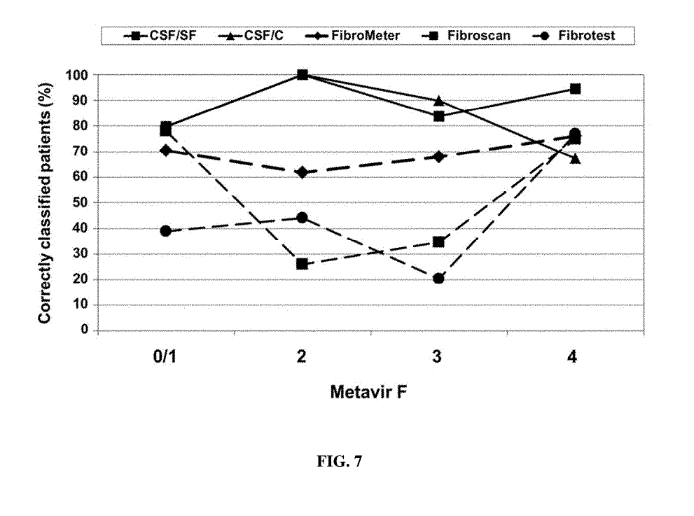

FIG. 7 shows the diagnostic accuracy of each fibrosis stage classification as a function of Metavir fibrosis stage in the validation set. Among single fibrosis tests, FibroMeter provided the most homogeneous profile with no significant differences among histological fibrosis stages (p=0.352). The new CSF/SF and CSF/C classifications provided better profiles than those of single fibrosis tests. However, the rate of well classified patients among cirrhotic patients was significantly higher with CSF/SF classification (94.5%) than with CSF/C classification (67.3%, p<0.001).

### Influencing Factors

In the whole study population, we performed a stepwise binary logistic regression including age, sex, biopsy length, Metavir F, and IQR/median as independent variables. The rate of well classified patients by CSF/SF classification was independently associated with the ratio IQR/median (1^{st} step, exp(β)=0,322), Metavir F (2^{nd} step, exp(β)=1.370), and age (3^{rd} step, exp(β)=0.976)

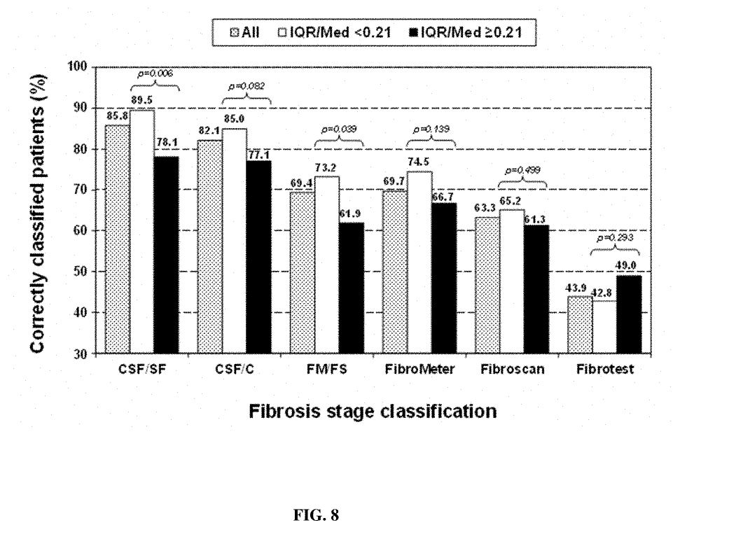

In the validation set, CSF/SF classification provided 89.5% diagnostic accuracy in patients with IQR/median <0.21 versus 78.1% in patients with IQR/median ≥0.21 (p=0.006). In the subgroup of patients with IQR/median <0.21, CSF/SF classification had the highest diagnostic accuracy (p=0.006 vs other classifications, FIG. 8).

1. A method for implementing an adapted patient care for an individual suffering from a liver fibrosis comprising:
determining in the individual the presence and severity of a liver fibrosis by:
(a) carrying out at least one non-invasive test resulting in a value;
(b) positioning the at least one test value in a class of a detailed classification of fibrosis stages or of necrotico-inflammatory activity grades based on population percentiles, wherein the detailed classification is obtained by:
carrying out at least one non-invasive test resulting in at least one value for each subject of a reference population;
classifying the subjects of the reference population into percentiles according to the test value obtained for said non-invasive test;
determining for each percentile of subjects of the reference population the associated fibrosis stage(s) or necrotico-inflammatory activity grade(s) according to a fixed minimal correct classification rate and a maximal number of fibrosis stage(s) or necrotico-inflammatory activity grade(s), thus allowing the grouping of stages or grades into new classes;
(c) assessing the presence and severity of a liver fibrosis, based on the class wherein said test value, has been positioned in step (b), and
implementing an adapted patient care for the individual depending on the severity of the liver fibrosis.

2. The method of claim 1, wherein the detailed classification is a detailed fibrosis classification wherein each class corresponds to less than or equal to 2 pathological fibrosis stages with reference either to the Metavir system or to the NASH-CRN scoring system or the detailed classification is a detailed necrotico-inflammatory activity classification wherein each class corresponds to less than or equal to 2 pathological activity grades.

3. The method of claim 1, wherein the non-invasive test comprises the measure of at least one data issued from Vibration Controlled Transient Elastography (VCTE), also known as Fibroscan.

4. The method of claim 1, wherein the non-invasive test comprises at least one combination score, obtained by mathematical combination of at least one biomarker, at least one clinical marker, at least one data resulting from a physical method and/or at least one score.

5. The method of claim 4, wherein said combination score is a test selected from the group consisting of ELF, FibroSpect™, APRI, FIB-4, Hepascore, Fibrotest™, CirrhoMeter™ and FibroMeter™, wherein:
ELF is a blood test based on hyaluronic acid, P3P, TIMP-1 and age;
FibroSpect™ is a blood test based on hyaluronic acid, TIMP-1 and A2M;
APRI is a blood test based on platelet and AST;
FIB-4 is a blood test based on platelet, ASAT, ALT and age;
Hepascore is a blood test based on hyaluronic acid, bilirubin, alpha2-macroglobulin, GGT, age and sex
Fibrotest™ is a blood test based on alpha2-macroglobulin, haptoglobin, apolipoprotein A1, total bilirubin, GGT, age and sex
FibroMeter™ and CirrhoMeter™ each are a blood test based on alpha2-macroglobulin, hyaluronic acid, prothrombin index, platelets, ASAT, ALAT, Urea, GGT, bilirubin, ferritin, glucose, age and/or sex.

6. The method of claim 4, wherein said combination score is a FibroMeter^{V3G}.

7. The method of claim 4, wherein said physical method is selected from the group consisting of Doppler-ultrasonography, elastometry ultrasonography, Vibration Controlled Transient Elastography (VCTE) also known as Fibroscan, Acoustic Radiation Force Impulse (ARFI), supersonic imaging, IRM, and MNR.

8. The method of claim 1, wherein said detailed classification is based on the discretization of the score results of a reference population into 40 percentiles of 2.5% of the population.

9. The method of claim 1, wherein the individual is at risk of suffering or is suffering from a condition selected from the group consisting of a liver impairment, chronic liver disease, a chronic hepatitis viral infection caused by hepatitis B, C or D virus, a hepatotoxicity, a liver cancer, a steatosis, an alcoholic liver disease (ALD), a non-alcoholic fatty liver disease (NAFLD), a non-alcoholic steatohepatitis (NASH), an autoimmune disease, a metabolic liver disease and a disease with secondary involvement of the liver.

10. The method of claim 8, wherein the individual is at risk of suffering or is suffering from a condition selected from the group consisting of a steatosis, a non-alcoholic fatty liver disease (NAFLD), a non-alcoholic steatohepatitis (NASH), an autoimmune disease, and a metabolic liver disease.

11. The method of claim 1, wherein the individual is determined to suffer from liver fibrosis at stage F≥1, with reference either to the Metavir system or to the NASH-CRN scoring system, and the adapted patient care consists in monitoring said individual by assessing the fibrosis severity at regular intervals.

12. The method of claim 1, wherein the individual is determined to suffer from liver fibrosis at stage F≥2, with reference either to the Metavir system or to the NASH-CRN scoring system, and the adapted patient care consists in administering without delay at least one therapeutic agent or starting a complication screening program for applying early prophylactic or curative treatment.

13. The method according to claim 12, wherein the at least one therapeutic agent is an antifibrotic agent selected from the group consisting of simtuzumab, GR-MD-02, stem cell transplantation (in particular MSC transplantation), Phyllanthus urinaria, Fuzheng Huayu, S-adenosyl-L-methionine, S-nitrosol-N-acetylcystein, silymarin, phosphatidylcholine, N-acetylcysteine, resveratrol, vitamin E, losartan, telmisartan, naltrexone, RF260330, sorafenib, imatinib mesylate, nilotinib, INT747, FG-3019, oltipraz, pirfenidone, halofuginone, polaorezin, gliotoxin, sulfasalazine, rimonabant and combinations thereof.

14. The method according to claim 12, wherein the at least one therapeutic agent is for treating the underlying cause responsible for liver fibrosis, and/or ameliorating or alleviating the symptoms or lesions associated with the underlying cause responsible for liver fibrosis, including liver fibrosis.

15. The method according to claim 14, wherein the underlying cause responsible for liver fibrosis is a viral infection and the at least one therapeutic agent is selected from the group consisting of interferon, peginterferon 2b (pegylated IFNalpha-2b), infliximab, ribavirin, boceprevir, telaprevir, simeprevir, sofosbuvir, daclatasvir, elbasvir, grazoprevir, velpatasvir, lamivudine, adefovir dipivoxil, entecavir, telbivudine, tenofovir, clevudine, ANA380, zadaxin, CMX 157, ARB-1467, ARB-1740, ALN-HBV, BB-HB-331, Lunar-HBV, ARO-HBV, Myrcludex B, GLS4, NVR 3-778, AIC 649, JNJ56136379, ABI-H0731, AB-423, REP 2139, REP 2165, GSK3228836, GSK33389404, RNaseH Inhibitor, GS 4774, INO-1800, HB-110, TG1050, HepTcell, TomegaVax HBV, RG7795, SB9200, EYP001, CPI 431-32 and combinations thereof.

16. The method according to claim 14, wherein the underlying cause responsible for liver fibrosis is excessive alcohol consumption and the at least one therapeutic agent is selected from the group consisting of topiramate, disulfiram, naltrexone, acamprosate and baclofen.

17. The method according to claim 14, wherein the underlying cause responsible for liver fibrosis is a non-alcoholic fatty liver disease (NAFLD) and the at least one therapeutic agent is selected from the group consisting of telmisartan, orlistat, metformin, pioglitazone, atorvastatin, ezetimine, vitamin E, sylimarine, pentoxyfylline, ARBs, EPL, EPA-E, multistrain biotic (L. rhamnosus, L. bulgaricus), simtuzumab, obeticholic acid, elafibranor (GFT505), DUR-928, GR-MD, 02, aramchol, RG-125, cenicriviroc CVC and combinations thereof.

18. The method of claim 1, wherein the individual is afflicted with NAFLD, said method comprising:
determining in the individual with NAFLD the presence and severity of a liver fibrosis by:
(a) carrying out at least one non-invasive test resulting in a value;
(b) positioning the at least one test value in a class of a detailed classification of fibrosis stages according to the NASH-CRN scoring system based on population percentiles, wherein the detailed classification is obtained by:
carrying out at least one non-invasive test resulting in at least one value for each subject of a reference population;
classifying the subjects of the reference population into percentiles according to the test value obtained for said non-invasive test;
determining for each percentile of subjects of the reference population the associated fibrosis stages according to the NASH-CRN scoring system according to a fixed minimal correct classification rate and a maximal number of fibrosis stage(s) according to the NASH-CRN scoring system, thus allowing the grouping of stages or grades into new classes;
(c) assessing in the individual with NAFLD the presence and severity of a liver fibrosis based on the class wherein said test value has been positioned in step (b), and
implementing an adapted patient care for the individual with NAFLD depending on the severity of the liver fibrosis.

19. The method of claim 18, wherein the presence and severity of a liver fibrosis in the individual with NAFLD is determined by:
(a) carrying out at least one FibroMeter^{V2G} resulting in a value;
(b) positioning the at least one test value in a class of a detailed classification of fibrosis stages according to the NASH-CRN scoring system based on population percentiles comprising 6 classes, namely F1±1, F1/2, F2/3, F3±1, F3/4, and F4;
(c) assessing in the individual with NAFLD the presence and severity of a liver fibrosis based on the class wherein said the FibroMeter^{V2G} value has been positioned in step (b).

20. The method of claim 18, wherein the presence and severity of a liver fibrosis in the individual with NAFLD is determined by:
(a) carrying out at least one Fibroscan, also known as VCTE, resulting in a value;
(b) positioning the at least one test value in a class of a detailed classification of fibrosis stages according to the NASH-CRN scoring system based on population percentiles comprising 7 classes, namely F0/1, F1±1, F1/2, F2/3, F3±1, F3/4, and F4;
(c) assessing in the individual with NAFLD the presence and severity of liver fibrosis based on the class wherein said the Fibroscan value has been positioned in step (b).
